(19) United States
(12) Patent Application Publication
(10) Pub. No.: US 2025/0032303 A1
(43) Pub. Date: Jan. 30, 2025

(54) DEVICES FOR USE WITH SURGICALLY CREATED ORIFICES

(71) Applicant: THE INSIDES COMPANY LIMITED, Auckland (NZ)

(72) Inventors: Gregory Brian O'Grady, Auckland (NZ); John Bilkey Davidson, Auckland (NZ); Robert Bruce Davidson, Auckland (NZ)

(21) Appl. No.: 18/598,171

(22) Filed: Mar. 7, 2024

Related U.S. Application Data
(63) Continuation of application No. 16/978,951, filed on Sep. 8, 2020, now abandoned.

(30) Foreign Application Priority Data
Mar. 8, 2018 (NZ) 740603
Aug. 7, 2018 (NZ) 744989
Aug. 8, 2018 (NZ) 745086

(51) Int. Cl. A61F 5/44; A61F 5/443; A61F 5/445

(52) U.S. Cl. CPC A61F 5/4407; A61F 5/4401; A61F 5/443; A61F 2005/4455

(57) ABSTRACT

A device for connecting and transferring contents between a surgically created first orifice and a surgically created second orifice is provided. The device comprises a hollow body member and first and second tubular legs depending from the hollow body, and being adapted for insertion into the first and second surgically created orifices respectively. The device further comprises a cap for closing the hollow body and completing a sealed pathway between the tubular legs for passively conveying contents between the first and second surgically created orifices. Sealing features of the device are provided that in combination operate to keep the contents away from unwanted skin areas and/or provide an easy-to-use device.

### CROSS REFERENCE TO RELATED APPLICATIONS

This application is a continuation of U.S. application Ser. No. 16/978,951, filed Sep. 8, 2020, which was the National Stage of International Application PCT/IB2019/051873, filed Mar. 8, 2019, the entireties of which are incorporated herein by reference.

### FIELD OF THE INVENTION

The present invention relates to a device suitable for use with surgically created orifices. More particularly, but not exclusively, the present invention relates to devices for use in connecting ostomies or stomas of a gastrointestinal tract that are surgically created.

### BACKGROUND OF THE INVENTION

Colorectal cancer is a cancer which develops from the colon or rectum and is currently the third most prevalent cancer worldwide. Ileostomies remain a cornerstone of colorectal cancer management, particularly for rectal cancer management. Ileostomy involves dividing the ileum (the distal small bowel) and bringing one or both ends onto the skin of a patient so that digestive contents can flow into a stoma bag. Traditionally, the digestive contents bypass the colon and are collected in the stoma bag and then emptied manually.

Loop ileostomy is a common type of ileostomy which is intended to temporarily divert digestive contents, while the gastrointestinal tract distal to the ileum heals after colorectal surgery. It is usually intended that the loop ileostomy will be reversed by re-connecting the divided ends of the bowel back together, once healing has occurred. A radiological test is usually performed before the reversal surgery, to confirm that healing has occurred and that the bowel is not leaking. Usually a patient requires at least 1-2 weeks for their gastrointestinal tract distal to the ileum to heal sufficiently to perform a radiological leak test and consider reversal surgery.

After a leak test, some patients may have an “early reversal” of their ileostomy, performed within 1-2 weeks after surgery.

However, it is more usual for ileostomy reversal surgery to occur around 6-12 weeks after surgery. It is generally not considered to be safe to perform the reversal surgery between 2-6 weeks after surgery, because of the presence of internal adhesions that increase surgical risk. If a patient is having chemotherapy, they may need to retain an ileostomy for many months until the chemotherapy is finished, before surgery can be safely performed.

As digestive contents are evacuated from the patient's body, patients may suffer from fluid, electrolyte and nutrient losses as ileostomy bypasses the colon which resorbs water, electrolytes and certain nutrients. As a result, patients may experience dehydration and subsequent renal injury, and may need to be readmitted to hospital for management with intravenous fluid replacement. Some patients have “high output” ileostomies, being at increased risk of dehydration and renal injury, and may therefore need to be additionally managed with medications that slow gut transit, and with oral rehydration solutions that may be poorly tolerated. Stoma patients often use a lot of hospital resources due to the additional management required, and the related readmissions due to stoma-related dehydration.

Ileostomy patients having chemotherapy, may also suffer excessive stoma output during chemotherapy, which leads to dose reductions, hospital readmissions, and suboptimal therapy. Leaving the colon without any internal nutrients for an extended duration may also increase the risk of “anterior resection syndrome”, which is poor bowel function after rectal cancer surgery. Anterior resection syndrome is associated with a poorer quality of life in colorectal cancer survivors.

When an ileostomy is reversed, patients may experience ileus (slow recovery of gut function), partly due to atrophy of the gut distal to the ileostomy due to the lack of nutrition. A loop ileostomy also depletes the bacterial microbiome that normally occupies the colon, which contributes to colonic health, and as a result patients undergoing reversal surgery may experience Clostridium difficile infections. Clostridium difficile infections prolong hospital stay, can cause serious illness, and may require additional treatments such as antibiotics, surgery or fecal transplants.

Another group of patients who may require a stoma bag are patients with enterocutaneous fistulas, which is an abnormal connection between the gut and the skin. An enterocutaneous fistula may arise as a complication of surgery, or due to a traumatic injury, or because of another disease process such as inflammatory bowel disease. Some neonates may get enterocutaneous fistulas or stomas because of a disease called necrotising enterocolitis, where a segment of gut becomes necrotic.

A type of stoma bag is usually placed over the enterocutaneous fistula to catch the digestive contents. Patients with fistulas often experience poor nutrition, and may become dependent on supplementary feeding such as parenteral nutrition, which is given through a vein. Parenteral nutrition is usually an expensive and risky treatment due to the possibilities of line infections and liver damage.

Furthermore, patients generally dislike having to wear and manage a stoma bag and the associated waste contents. Patients may also need to wake overnight to empty their ileostomies, disrupting sleep patterns. It may be desirable to make waste management of the stoma bag easier for patients, carers and medical practitioners.

There is a need for a device that requires no stoma bag at all, but rather contents simply flow from one portion to another. In some applications, such device could serve as a temporary solution to stoma losses, placed after the radiological leak test is performed, until the time of stoma reversal surgery. When used in this way, such a device could reduce or eliminate many of the adverse consequences of temporary stomas.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

For the purpose of this specification, where method steps are described in sequence, the sequence does not necessarily mean that the steps are to be chronologically ordered in that sequence, unless there is no other logical manner of interpreting the sequence.

### OBJECT OF THE INVENTION

It is an object of the invention to provide device suitable for use with surgically created orifices which overcomes or at least partially ameliorates some of the abovementioned disadvantages or which at least provides the public with a useful choice.

### Definitions

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprises’ or ‘comprised’ or ‘comprising’ is used in relation to the apparatus or to one or more steps in a method or process.

As used herein the term “and/or” means “and” or “or”, or both.

As used herein “(s)” following a noun means the plural and/or singular forms of the noun.

When used in claim and unless otherwise stated, the word ‘for’ is to be interpreted to mean only ‘suitable for’ and not, for example, specifically ‘adapted’ or ‘configured’ for the specific purpose that is stated.

### BRIEF DESCRIPTION OF THE INVENTION

According to a first aspect the invention broadly comprises a connecting device for connecting a surgically created first orifice with a surgically created second orifice and transferring contents from the surgically created first orifice to the surgically created second orifice, the device comprising:
a first elongate tubular member configured to, in use, be inserted into the surgically created first orifice,
a second elongate tubular member configured to, in use, be inserted into the surgically created second orifice,
an intermediate hollow body member having a body inlet and a body outlet,
and
wherein the first elongate tubular member is in fluid communication with and extends away from the body inlet, the first elongate tubular member having an inlet located at a distal end away from the intermediate hollow body member, and
wherein the second elongate tubular member is in fluid communication with and extends away from the body outlet, the second elongate tubular member having an outlet located at a distal end away from the intermediate hollow body member, and
wherein the intermediate hollow body member extends between the first and second elongate tubular members, and
wherein the intermediate hollow body member includes a body opening, and the connecting device includes a cap for closing said body opening to complete a sealed pathway between the first elongate tubular member and the second elongate tubular member for passive transfer of contents from the first orifice to the second orifice.

According to another aspect said cap further comprises a shell configured to, in use, encompass and defines a volume of space surrounding said surgically created first and second orifices, and wherein said volume of space is isolated from said pathway between the first elongate tubular member and the second elongate tubular member.

According to another aspect the shell is rigid or semi-rigid and adapted to exert a downward pressure to keep the first and second elongate tubular members within the surgically created first and second orifices when the device is attached to a patient.

According to another aspect said cap comprises a pathway seal on an inner surface of the cap adapted to seal the body opening.

According to another aspect the volume of space surrounding said surgically created first and second orifices is isolated from the pathway between the first elongate tubular member and the second elongate tubular member so that contents being transferred from the surgically created first orifice to the surgically created second orifice do not leak into the surrounding volume of space.

According to another aspect said cap is separate and removable and when removed allows access to an internal surface of the intermediate hollow body member.

According to another aspect the removable cap is connected to the hollow body member with a magnetic connection, or a press fit connection.

According to another aspect said cap and said shell are integrally formed.

According to another aspect the intermediate hollow body member and the first and second elongate tubular members are integrally formed.

According to another aspect the first and second elongate tubular members are separate and removable from the intermediate hollow body member.

According to another aspect the first tubular member and the second tubular member are flexible and resilient.

According to another aspect said device further comprises a plate structure for removable attachment to skin of a patient and around the surgically created first and second orifices, the plate structure comprising:
a membrane having a first side and a second side that are opposite to one another, the first side of the membrane is configured to be attached to a skin of a patient using an adhesive; and
a first plate coupling member attached to the second side of the membrane; and
wherein, the first plate coupling member is configured to be connected to a cooperating first coupling member.

According to another aspect the plate structure further comprises a protective layer which is adapted to be peeled off during use to reveal an adhesive coating on the first side of the membrane.

According to another aspect the membrane comprises at least one aperture at or near the centre of the membrane, wherein the size of the aperture is same or substantially the same as the size of orifice(s) over which the plate structure is intended to be used.

According to another aspect the first coupling member is a shell coupling member of the shell adapted to connect the shell to the plate structure.

According to another aspect the first plate coupling member and the first coupling member comprises complementary features in the form of a groove and a protrusion.

According to another aspect further comprising a pad that is fluid resistant and/or fluid absorbent and is adapted to cover at least one orifice formed on a patient's body during surgery.

According to another aspect the pad comprises a lattice spring biasing member that is adapted to increase the flexibility of the pad.

According to another aspect a pad coupling member on the pad is adapted to connect the pad to the plate structure.

According to another aspect the first plate coupling member forms a watertight coupling with the first coupling member.

According to another aspect the first plate coupling member is a clip in the form of a ring that is adapted to clip with the first coupling member.

According to another aspect the shell further comprises a flat rim extending from a perimeter of the shell, the flat rim having an underside configured to sit against a patient's skin.

According to another aspect the underside of the flat rim comprises an adhesive to adhere the shell to the patient's skin.

According to another aspect the distal end of at least one of said first tubular member and said second tubular member comprises a cage feature having cage supports and cage openings between neighbouring cage supports to allow the contents to pass.

According to another aspect the cage feature is tapered so that the cage feature reduces in diameter as it extends away from the hollow body member.

According to another aspect one or both tubular members comprise a longitudinal guide member extending from the distal end of the tubular member.

According to another aspect the cage supports are equally distributed round a longitudinal axis of the cage feature.

According to another aspect the longitudinal guide members are flexible.

According to another aspect the longitudinal guide members have a diameter between 5 and 12 mm.

According to another aspect the longitudinal guide members have a length between 20 and 60 mm.

According to another aspect at least one of said first tubular member and said second tubular member comprises an internal rib protruding from a sidewall of the tubular member to increase the wall thickness and stiffness of the tubular member.

According to another aspect at least one tubular member comprises a flared anchoring portion having a concave outward surface in a relaxed state.

According to another aspect said flared portion is in the form of an inflatable cuff located at or towards the distal end of a respective tubular member.

According to another aspect the distal end of at least one of said first elongate tubular member and said second elongate tubular member is bevelled, to define an oblique aperture.

According to another aspect one of the first and second tubular members is longer than the other of the first and second tubular members so that the longer tubular member is inserted further into the respective surgically created orifice.

According to another aspect the first and second tubular members each comprises a size between approximately 8 French and 12 French.

According to another aspect the first and second tubular members each comprises a size between approximately 32 French and 42 French.

According to another aspect the first tubular member comprises a diameter greater than the second tubular member.

According to another aspect silicone, rubber, latex or plastic form one or more of the:
a) first elongate tubular member,
b) second elongate tubular member,
c) intermediate hollow body member,
d) longitudinal guide member.

According to another aspect the first tubular member, second tubular member and/or the hollow body member comprises a flexible mesh having shape memory, and a wall associated with the mesh and collapsible for insertion and expandable for retention in the surgically created orifices.

According to another aspect the flexible mesh is formed as a nitinol alloy mesh.

According to another aspect the invention further comprises a method of connecting a surgically created first orifice with a surgically created second orifice, the method comprising:
providing a connecting device as described in previous clauses,
inserting the inlet of the first elongate tubular member into the surgically created first orifice and the outlet of the second elongate tubular member into the surgically created second orifice so that the surgically created first and second orifices are connected by the device for passive transfer of contents from the surgically created first orifice to the surgically created second orifice.

According to another aspect the method further comprises positioning and adhering a first side of a membrane of the plate structure to the skin of a patient.

According to another aspect the method further comprises positioning a shell over the surgically created first and second orifices to substantially encompass and define a volume of space surrounding said surgically created first and second orifices, and
coupling the shell to the plate structure by coupling the plate coupling member to the shell coupling member.

According to another aspect the method further comprises positioning the cap over the body opening to close the pathway between the first elongate tubular member and the second elongate tubular member.

According to another aspect the invention further comprises a method of connecting a surgically created first orifice with a surgically created second orifice with a connecting device as described in previous clauses, wherein the method includes the steps of:
collapsing the first tubular member or second tubular member before inserting the respective member into the respective orifice, and
releasing the respective member after insertion and the respective member expanding such that it is retained within the respective orifice.

According to another aspect the invention broadly comprises a connecting device for receiving digestive contents from a surgically created orifice, the device comprising:
a tubular member having a body opening and an inlet located at a distal end away from the body opening, the inlet adapted to be inserted into the surgically created orifice; and
a cap for closing said body opening forming an inner chamber; and
wherein, the distal end of the tubular member comprises a cage feature having cage supports and cage openings between neighbouring cage supports to allow the contents to pass.

According to another aspect said device further comprises a plate structure for a removable attachment to a skin of a patient and around the orifice formed on a patient's body during surgery, the plate structure comprising:
a membrane having a first side and a second side that are opposite to one another, the first side of the membrane is configured to be attached to a skin of a patient using an adhesive; and
a first plate coupling member attached or configured to be attached to the second side of the membrane; and
wherein, the first plate coupling member is configured to be connected to a cooperating first coupling member.

According to another aspect said cap further defines a shell that substantially encompasses and defines a volume of space surrounding the surgically created orifice, such that the volume of space is isolated from the inner chamber and the contents entering the tubular member do not leak into the surrounding volume of space.

According to another aspect the shell comprises a shell coupling member adapted to connect the shell to the plate structure.

According to another aspect the first plate coupling member and the first coupling member comprises complementary features in the form of a groove and a protrusion.

According to another aspect the device further comprises a pad that is fluid resistant and/or fluid absorbent and is adapted to cover at least one orifice formed on a patient's body during surgery, and the pad comprises a lattice spring biasing member that is adapted to increase the flexibility of the pad.

According to another aspect said cap is separate and removable which when removed allows access to an internal surface of the tubular member.

According to another aspect the cage feature is tapered so that the cage feature reduces in diameter as it extends away from the body opening.

According to another aspect the tubular member comprises a longitudinal guide member.

According to another aspect the longitudinal guide member has a diameter between 5 and 12 mm.

According to another aspect the longitudinal guide member has a length between 20 and 60 mm.

According to another aspect the longitudinal guide member is flexible.

According to another aspect the tubular member comprises an internal rib protruding from a sidewall of the tubular member to increase the wall thickness and stiffness of the tubular member.

According to another aspect the tubular member comprises a flexible mesh having shape memory, and a wall associated with the mesh and collapsible for insertion and expandable for retention in the surgically created orifices.

According to another aspect the flexible mesh is formed as a nitinol alloy mesh.

According to another aspect the tubular member is flexible and resilient.

According to another aspect the shell further comprises a flat rim extending from a perimeter of the shell, the flat rim having an underside configured to sit against a patient's skin.

According to another aspect the underside of the flat rim comprises an adhesive to adhere the shell to the patient's skin.

According to another aspect the tubular member comprises a size between approximately 8 French and 12 French adapted for neonatal patients.

According to another aspect tubular member comprises a size between approximately 32 French and 42 French adapted for adult patients.

According to another aspect the invention broadly comprises a connecting device for receiving digestive contents from a surgically created orifice, the device comprising:
a tubular member having a body opening and an inlet located at a distal end away from the body opening, the inlet adapted to be inserted into the surgically created orifice; and
a shell comprising an aperture, the shell having an internal and external side that are opposite one another, the shell substantially encompasses and defines a volume of space surrounding the surgically created orifice,
wherein, the shell is connected to the tubular member at or towards the body opening so that digestive contents exits the body opening on the external side of the shell,
wherein, the shell comprises a first shell coupling member configured to be connected to a cooperating first coupling member.

According to another aspect said device further comprises a plate structure for removable attachment to skin of a patient and around the surgically created orifice, the plate structure comprising:
a membrane having a first side and a second side that are opposite to one another, the first side of the membrane is configured to be attached to a skin of a patient using an adhesive; and
a first plate coupling member attached to the second side of the membrane; and
wherein, the first plate coupling member is configured to be connected to the first shell coupling member.

According to another aspect the first shell coupling member is located at the lower perimeter of the shell, to couple the lower perimeter of the shell to the plate structure.

According to another aspect the shell comprises a second shell coupling member configured to be connected to a cooperating second coupling member.

According to another aspect the invention further comprises a stoma bag adapted to cover the body opening to receive digestive contents from the body opening, and the stoma bag comprises a bag coupling member adapted to be connected to the second shell coupling member.

According to another aspect the first shell coupling member and the second shell coupling member have substantially the same diameter and the first shell coupling member and the second shell coupling member is a double-sided, integrated coupling structure.

According to another aspect the first and second shell coupling members form a watertight coupling with the first and second cooperating coupling members.

According to another aspect the first and second plate coupling members and the first and second cooperating coupling members have complementary features in the form of grooves and protrusions.

According to another aspect the shell is rigid or semi-rigid and adapted to exert a downward pressure to keep the tubular member within the surgically created orifice when the device is attached to a patient.

According to another aspect the device further comprises a cap or plug to close the shell aperture.

According to another aspect the distal end of the tubular member comprises a cage feature having cage supports and cage openings between neighbouring cage supports.

According to another aspect the cage feature is tapered so that the cage feature reduces in diameter as it extends away from the body opening.

According to another aspect the tubular member comprises a longitudinal guide member extending from the distal end of the tubular member.

According to another aspect the longitudinal guide member has a diameter between 5 and 12 mm.

According to another aspect the longitudinal guide member has a length between 20 and 60 mm.

According to another aspect the longitudinal guide member is flexible.

According to another aspect the tubular member comprises an internal rib protruding from a sidewall of the tubular member to increase the wall thickness and stiffness of the tubular member.

According to another aspect the tubular member comprises a flared anchoring portion having a concave outward surface in a relaxed state.

According to another aspect said anchoring means in the form of an inflatable cuff located at or towards the distal end of the tubular member.

According to another aspect the distal end of the tubular member is bevelled, to define and an oblique aperture.

According to another aspect the tubular member comprises a flexible mesh having shape memory, and a wall associated with the mesh and collapsible for insertion and expandable for retention in the surgically created orifices.

According to another aspect flexible is formed as a nitinol alloy mesh.

According to another aspect the tubular member is flexible and resilient.

According to another aspect the shell further comprises a flat rim extending from a perimeter of the shell, the flat rim having an underside configured to sit against a patient's skin.

According to another aspect the underside of the flat rim comprises an adhesive to adhere the shell to the patient's skin.

According to another aspect the tubular member comprises a size between approximately 8 French and 12 French adapted for neonatal patients.

According to another aspect tubular member comprises a size between approximately 32 French and 42 French adapted for adult patients.

According to another aspect the invention further comprises a method of connecting a device to a surgically created orifice to receive digestive contents, the method comprising:
providing a connecting device,
inserting the inlet of the tubular member into the surgically created orifice.

According to another aspect connecting a device to a surgically created orifice to receive digestive contents as described in the previous clause, wherein the method comprises a step of positioning and adhering a first side of a membrane of the plate structure as described in previous clauses to the skin of a patient.

According to another aspect the method further comprises:
positioning a shell over the surgically created orifices to substantially encompass and define a volume of space surrounding said first and second surgically created orifices, and
coupling the shell to the plate structure by coupling the first plate coupling member to the shell coupling member.

According to another aspect the method further comprises positioning the stoma bag over the body opening to receive digestive contents from the body opening, and
coupling the stoma bag to the plate structure by coupling the second plate coupling member to the bag coupling member.

According to another aspect the method further comprises collapsing the tubular member before inserting the member into the orifice, and
releasing the member after insertion such that it expands and is retained within the orifice.

According to another aspect the method further comprises transferring contents from the first orifice to the second orifice, the device comprising a tubular body having an enclosing wall, the body having an external surface and an internal surface, and extending from a first open end to a second open end;
wherein the body is flexible and has a shape memory;
wherein at least part of the body is substantially elliptical or circular in cross-section;
wherein the body is constructed of a material allowing the device to collapse for insertion and then expand or tend to expand towards an initial non-collapsed state for retention;
wherein the first open end is configured to be inserted to the first orifice and the second open end is configured to be inserted to the second orifice to create a pathway for contents between the two surgically created orifices.

According to another aspect the body portion comprises a flexible mesh having shape memory, and the wall is associated with the mesh to form the tubular body for contents to pass between the first and second orifices.

According to another aspect the flexible mesh comprises a series of filaments extending at least a majority of the body, the series of filaments being bonded with said wall and collapsible for insertion and expandable for retention in the surgically created orifices.

According to another aspect the flexible mesh is formed of a nitinol alloy mesh.

According to another aspect the body portion comprises a bend to form a U-shape, V-shape or another arcuate shape and form a first and second limb on either side of the bend.

According to another aspect the first limb and second limb are adapted to be forced towards each other to be inserted into the first and second orifices, and tend to move away from each other when released to retain in the surgically created first and second surgically orifices.

According to another aspect at least one of the first and second open ends comprises a flared anchoring portion configured to be inserted into and retained in the surgically created first and second surgically orifices.

According to another aspect the body portion comprises at least one removable cap which when removed allows access to the internal surface of the body portion.

According to another aspect the body portion comprises a hook or loop attachment member on the external surface, the attachment member being suitable to facilitate attachment to an ancillary device.

According to another aspect the body portion is formed of silicone, rubber, latex or plastic.

Other aspects of the invention may become apparent from the following description which is given by way of example only and with reference to the accompanying drawings.

For purposes of the description hereinafter, the terms “upper”, “lower”, “right”, “left”, “vertical”, “horizontal”, “top”, “bottom”, “lateral”, “longitudinal” and derivatives thereof shall relate to the invention as it is oriented in the drawing figures. However it is to be understood that the invention may assume various alternative variations, except where expressly specified to the contrary. It is also to be understood that the specific devices illustrated in the attached drawings, and described in the following specification are simply exemplary embodiments of the invention. Hence specific dimensions and other physical characteristics related to the embodiments disclosed herein are not to be considered as limiting.

### BRIEF DESCRIPTION OF THE DRAWINGS

The invention will now be described by way of example only and with reference to the drawings in which:

FIG. 1 shows a front elevation view of a connecting device of the first preferred embodiment of the invention in a relaxed state.

FIG. 2a shows a front elevation view of a connecting device of the second preferred embodiment of the invention in a relaxed state.

FIG. 2b shows a front elevation view of a connecting device of the second preferred embodiment with an optional attachment means, optional protrusions and a first type of optional cap.

FIG. 2c shows a partially exploded view a connecting device showing the cap being removed from the body portion.

FIG. 2d shows a front elevation partially exploded view of a connecting device of the second preferred embodiment of the invention with a cap arrangement comprising two optional caps.

FIG. 3 shows a perspective view of a connecting device of the third preferred embodiment of the invention in a relaxed state.

FIG. 4 shows a perspective view of a connecting device of the fourth preferred embodiment of the invention in a relaxed state.

FIGS. 5a-5b show a preferred method of using of the connecting device of the present invention.

FIG. 6a shows a sectional top perspective view of the covering device coupled with a plate structure.

FIG. 6b shows an exploded view of FIG. 6a.

FIG. 6c shows an exploded view of a kit of parts comprising a covering device and plate structure.

FIG. 7a is a perspective view of another preferred embodiment.

FIG. 7b is an exploded perspective view of the device of FIG. 7a.

FIG. 8 is a perspective view of another preferred embodiment including an integrated cap.

FIG. 9 is an exploded perspective view of the preferred device of FIG. 8.

FIG. 10 is a cross section view of a further preferred device showing an inflatable securing cuff.

FIG. 11 is a underside of the device of FIG. 10 having an integrated cap.

FIG. 12 is a perspective view of the device of FIG. 10.

FIG. 13 is a perspective partial view, showing an introducer.

FIG. 14 is a perspective view of an alternative device, showing bevelled leg ends.

FIG. 15 is a perspective view showing leg ends including a cage feature.

FIG. 16 is a perspective view showing leg ends including longitudinal guide members.

FIG. 17 is a cross section view of legs having a thickened wall region.

FIG. 18a is a schematic showing a device inserted through to dense facia.

FIG. 18b is a schematic showing a device with cage and longitudinal guide member features inserted through to dense facia.

FIG. 19 is a perspective view of a one-legged device with a stoma bag.

FIG. 20 is an exploded view of the device in FIG. 19.

FIG. 21a is a schematic of a shell with an aperture.

FIG. 21b is a schematic of a shell with a plug.

FIG. 21c are schematics showing a device with a tube leading away from the stoma.

FIG. 22 is a perspective view showing a device with a flat rim.

### DETAILED DESCRIPTION OF PREFERRED EMBODIMENTS

The following description will describe the invention in relation to examples and/or drawings. The invention is in no way limited to the example(s) and/or drawing(s) as they are purely to exemplify the invention only and that possible variations and modifications would be readily apparent without departing from the scope of the invention described in the specification and/or defined in the claims.

### First Preferred Embodiment

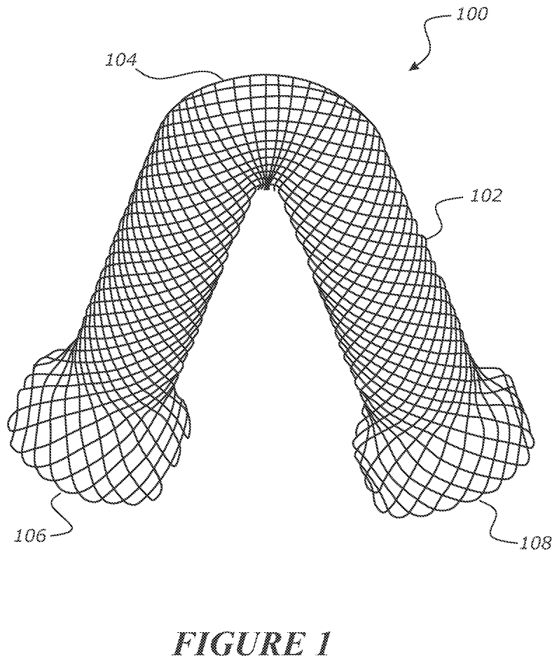

With reference to FIGS. 1-18B, there is shown several preferred embodiments of a connecting device of the present invention.

FIG. 1 shows a front elevation view of a connecting device of the first preferred embodiment of the invention in a relaxed state. The connecting device is suitable for connecting a first ostomy with a second ostomy and transferring the contents from the first ostomy to the second ostomy.

As it can be seen from FIG. 1, the connecting device (100) comprises a body portion (102) having an enclosing wall, that is tubular and has a curved portion (104). There is a first open end (106) and a second open end (108).

The body portion (102) is made out of a material that is flexible and elastic and therefore has a shape memory and/or resilience, i.e. the body portion (102) has a property to recover to its original curved shape. In its relaxed state, the body portion (102) may be a U-shape or a V shape or any other arcuate shape.

As shown in FIG. 1, the first open end (106) and the second open end (108) are flared/slight funnel and therefore the internal diameter of each of those flared ends are larger than the internal diameter of the body portion (102) that is not flared. The flaring of the first open end (106) and second open end (108) assist in anchoring of the connecting device (100) when the device (100) is inserted inside ostomies or stomas.

Such flaring also serve in sealing the inner wall of the orifice or lumen so that leaks of fluid or intestinal content do not occur around the edges of the connecting device (100). This will be discussed later in more detail.

As shown in FIG. 1, the body portion (102) of this first preferred embodiment of the connecting device is a hollow, generally cylindrical covered mesh structure. The structure of the body portion (102) includes a series of wires or filaments, which preferably extend at least to a majority of the body portion (102), and most preferably the entire body portion (102) as shown.

The filaments of the mesh structure are preferably elastic-shape memory alloy wires, for example, those made for elastic materials such as nitinol. Alternatively, the body portion (102) is formed of a silicon covered nitinol. Other materials may include shape memory polymers or metals, or simply elastic material(s), or other material(s) with some ability to collapse for insertion then expand for retention.

The body wall is associated with the mesh such that when the mesh collapses and expands, the wall will also moves to provide a desired body profile. It is anticipated that the flexible mesh is located within or exterior to the wall of the body. It is also anticipated that the flexible mesh is located between the body wall and another layer or any other lay-up which allows the body to be associated with the mesh.

Preferably, the wall is bonded to the wall so when the mesh expands or contracts, the wall moves with the mesh.

The resulting structure is a tube through which intestinal contents can pass, but which is also resilient giving the tube an ability to hold its shape against in its natural state, and/or an ability to be compressed/collapsed prior to deployment. The filaments may be continuously woven together in a braided form, however the structure is not limited to woven structures, and includes other similar fabrications such as but not limited to welding, crocheting, knitting, tying, suturing, or any other suitable methods for producing such mesh or interconnected structures.

### Second Preferred Embodiment

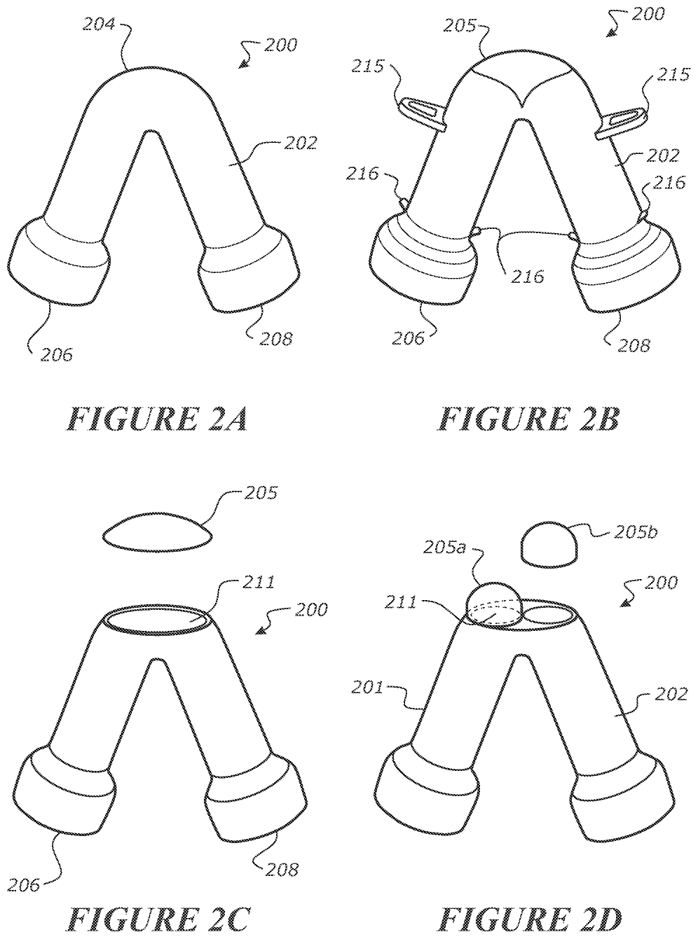

FIG. 2a shows a front elevation view of a connecting device of the second preferred embodiment of the invention in a relaxed state.

Similar to the first preferred embodiment shown in FIG. 1, the connecting device (200) according to this second preferred embodiment comprises a body portion (202) that is tubular and has a curved portion (204). There is a first open end (206) and a second open end (208).

The body portion (202) is made out of a material that is flexible and elastic and therefore has a shape memory and/or resilience, i.e. the body portion (202) has a property to recover to its original curved shape. In its relaxed state, the body portion (202) may be of a U-shape or a V-shape or any other arcuate shape.

The first open end (206) and the second open end (208) are flared/slight funnel and therefore the internal diameter of each of those flared ends are larger than the internal diameter of the body portion (202) that is not flared. The flaring of the first open end (206) and second open end (208) assist in anchoring of the connecting device (200) when the device (200) is inserted inside ostomies or stomas on the body of a patient. Such flaring also serve in sealing the inner wall of the orifice or lumen so that leaks of fluid or intestinal content do not occur around the edges of the connecting device (200).

Unlike, the embodiment of the device (200) as shown in FIG. 1, the body portion (202) of the connecting device (200) is not formed as a mesh reinforced structure, but instead formed as a non-mesh tubular structure. Some examples of suitable construction material for the body portion (202) are medical grade silicon, silicon rubber, polyurethane, PVC or latex rubber. Other materials may include shape memory polymers or metals, or simply elastic material(s), or other material(s) with some ability to collapse for insertion then expand for retention. The body portion (202) or part of the body portion (202) of the device (200) may be either transparent or translucent or opaque.

The device (200) may optionally comprise one or more protrusions (216) above the flared open ends (206 and 208) as shown in FIG. 2b. As mentioned above, the flaring of the first open end (206) and second open end (208) assist in anchoring of the connecting device (200) on the body orifices or stomas. These optional protrusions (216) can further assist with anchoring and facilitate a stronger grip. Instead of both ends (206 and 208), the protrusions (216) may optionally be formed above only one of the first open end (206) and second open end (208). The protrusions (216) may even be in a form of circumferential protrusions (not shown), i.e., they may extend around the circumference on the external surface of the body portion just above the flared open ends (206 and 208).

As shown in FIG. 2b, the device (200) may optionally comprise at least one cap (205) at or near the apex of the curved portion and/or one or more attachment means (215) on the external surface of the body portion (202).

FIGS. 2b-2d show the several examples of the cap arrangements that could be used in the present invention. Preferably, the cap (205) is a removable magnetic cap. Preferably, the part of the body portion (202) that engages with the cap (205) is also made out of a magnetic material so that the cap (205) can be magnetically attached and detached from the body portion (202).

However, other alternative means for the engagement of the cap (205) to the body portion (202) is equally possible, such as but not limited to engagement via hinged connection, threaded connection, biasing means, fasteners or a combination thereof. The cap (205) or at least a portion of the cap (205) may even be of bung-type, i.e., constructed of a flexible elastic material, so that at least a portion of the cap (205) can be compressed or squeezed to enable it to pass through the entry port of the inside portion (211) and once inserted, the compressed portion of the cap (205) can self-expand against the inner surface of the body portion (202) thereby creating a seal.

Having a cap (205) allows access to the inside portion (211) of the body portion (202). Having such access to the inside of the body portion (202) can assist in cleaning up of the inside portion (211) of the device (200) and releasing or flushing of any blockages or potential blockages from the inside portion (211) of the device (200) when the device (200) is implanted in the body of the patient.

Instead of one cap, the device (200) may have multiple caps, for example, two caps (205a, 205b), such as the one shown in FIG. 2d. In that embodiment, the first cap (205a) allows access to the inside portion (211) of the left side (201) of the device (200) and the cap (205b) allows access to the inside portion (211) of the right side (203) of the device (200). Having such access can assist in cleaning up of the inside portion (211) of the left side (201) and right side (203) or both of the device (200) and releasing or flushing of any blockages or potential blockages from inside portion (211) of the device (200) when the device (200) is implanted in the body of a patient. Preferably, the caps (205a, 205b) are removable magnetic caps. Preferably, the parts of the body portion (202) engaging with the caps (205a, 205b) are made out of a magnetic material so that the caps (205a, 205b) can be magnetically attached and detached from the body portion (202). However, other alternative means for the engagement of the caps (205a, 205b) to the body portion is equally possible such as but not limited to engagement via hinged connection, threaded connection, biasing means, fasteners or a combination thereof. Alternatively, at least one of the caps (205a, 205b) or portion it may be of bung-type as described above.

It will be appreciated that the attachment of each cap is preferred to be at least leak resistant.

As shown in FIG. 2b, the connecting device (200) may optionally include attachments means (215) in the form of a loop mainly for the connection to an ancillary device (not shown), such as a stoma belt, having corresponding means that is suitable for attaching to the attachment means (215). The attachments means (215) can be any other suitable attachment means for attaching to ancillary devices such as but not limited to hooks, clips, hook and loop fasteners etc.

The connecting device (100) of FIG. 1 having a mesh reinforced structure may equally be designed to include protrusion(s) above the flared open end(s) (106, 108) and/or at least one cap and/or at least one attachments means similar to the ones described above with reference to FIGS. 2b-2d. The cap may be of magnetic or bung-type or any other suitable type as described above and is preferably located at the apex of the mesh structure of the body portion (102). Similarly, the device (100) may also be designed to include one or more attachment means. Similar to the ones as described above, the attachment means of device (100) may be formed as part of the same mesh structure that forms the body portion (202) or could be another structure that is connected to the body portion by any suitable means.

As mentioned above, the cap (205) and/or attachments means (215) and/or protrusions (216) are purely optional. In a less complex design where caps (205) is/are not present, it is still possible to flush the contents causing blockages or potential blockages via alternative techniques. For example, a patient may be given a per-oral solution (such as oral ‘gastrografin’ or polyethylene glycol) which can dilute and flush impacted gastrointestinal blockages). Alternatively, it is also possible to remove and replace the device (100, 200) altogether in the event of blockages or potential blockages. Alternatively, since, the body portion (100, 200) of the device (100, 200) is made out of a flexible material, squeezing by applying external force to the device (102, 202) from outside, or vibration may also assist in clearing up of any blockages or potential blockages. Optionally, the lining of the device (100, 200) may be coated with a lubricious material or lubricious liner to aid passage of the contents and to reduce the chance of blockages. For example, polytetrafluoroethylene (or PTFE) or suitable nanoparticles or biocompatible materials may be used as a lubricious material.

### Third and Fourth Preferred Embodiments

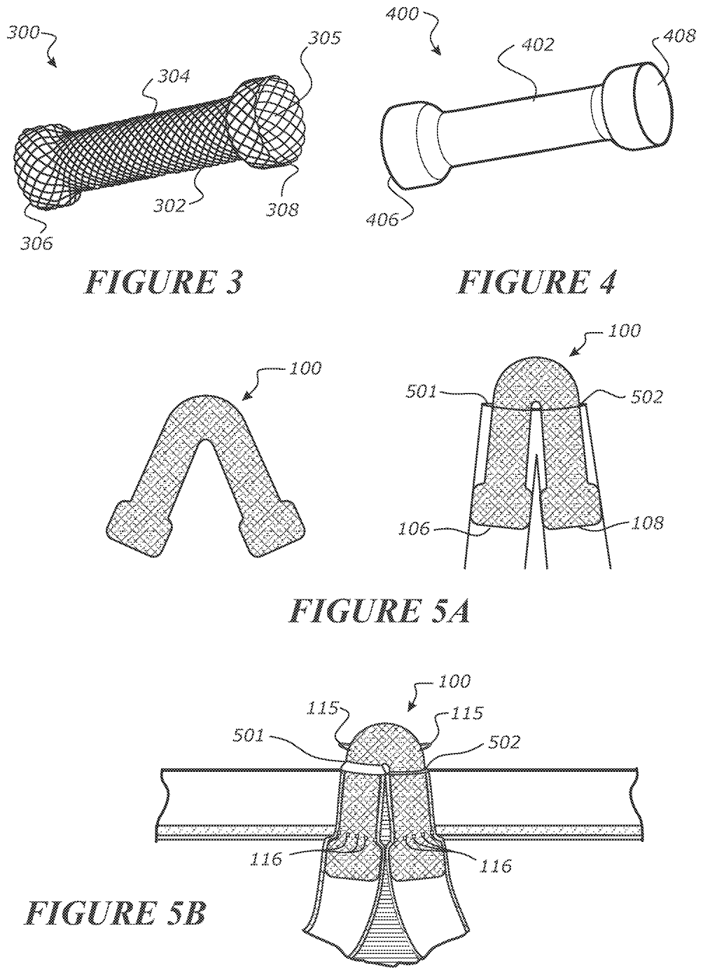

FIG. 3 and FIG. 4 show perspective views of a connecting device (300) of a third preferred embodiment and a fourth preferred embodiment of the invention respectively, in a relaxed state.

As shown in FIG. 3, the body portion (302) of the device (300) of the third preferred embodiment is tubular in shape and has an external surface (304), an internal surface (305), and a longitudinally opposed first and second open ends (306 and 308).

The body portion (302) is made out of a material that is flexible and elastic and therefore has a shape memory and/or resilience, i.e. the body portion (302) has a property to recover to its original elongated shape.

The first open end (306) and the second open end (308) are flared/slight funnel and therefore the internal diameter of each of those flared ends are larger than the internal diameter of the body portion (302) that is not flared. The flaring of the first open end (306) and second open end (308) assist in anchoring of the connecting device (300) on when the device (300) is inserted inside ostomies or stomas on the body of a patient. Such flaring also serve in sealing the inner wall of the orifice or lumen so that leaks of fluid or intestinal content do not occur around the edges of the connecting device (300).

Similar to the device (100) of the first preferred embodiment, the body portion (302) of this third preferred embodiment of the connecting device (300) is a hollow, generally cylindrical mesh reinforced structure. The structure of the body portion (302) includes a series of wires or filaments, which preferably extend at least to a majority of the length of the body portion (302), and most preferably the entire length of the body portion (302) as shown.

The filaments of the mesh structure are preferably elastic-shape memory alloy wires, for example, those made for elastic materials such as nitinol. Alternatively, the body portion (102) is formed of a silicon covered nitinol. Other materials may include shape memory polymers or metals, or simply elastic material(s), or other material(s) with some ability to collapse for insertion then expand for retention. The filaments may be continuously woven together in a braided form, however the structure is not limited to woven structures, and includes other similar fabrications such as but not limited to knitting, welding, crocheting, suturing, tying, or any other suitable methods for producing such mesh or interconnected structures.

As shown in FIG. 4, the device (400) of the fourth preferred embodiment is substantially similar to the device (300) of the third preferred embodiment described above and therefore most of the description above with reference in relation to device (300) applies equally to device (400). However, unlike the embodiment of the device (300) as shown in FIG. 3, the body portion (402) of the connecting device (400) does not include a mesh reinforcing structure, but instead formed as a non-mesh tubular structure. Some examples of suitable construction material for the body portion (402) are medical grade silicon, silicon rubber, polyurethane, PVC or latex rubber. Other materials may include shape memory polymers or metals, or simply elastic material(s), or other material(s) with some ability to collapse for insertion then expand for retention. The body portion (402) or part of the body portion (402) of the device (400) may be either transparent or translucent or opaque.

Although, not shown, the devices (300 and 400) of the third and fourth preferred embodiments may optionally include protrusion(s) and/or cap(s) and/or one or more attachment means(s) in a similar manner as described above with reference to FIGS. 2b-2d. The cap(s) may be positioned on the external surface at or near the middle of the elongated body portion (302 and 402) such that when the devices (300 and 400) are bent to an arcuate shape such that a U shape or substantially U shape, the cap appears at or near the apex of the curved portion. Similarly, one or more attachment means may be positioned between flared portion(s) and the middle of the elongated body portion on the external surface.

During use (i.e. during implant), the devices (300 and 400) of the third and fourth preferred embodiments are configured to be bent in an arcuate shape such as U-shape or V-shape or substantially U-shape or V-shape by applying an external force. When bent, the device (300) of the third preferred embodiment may look similar to the device (100) of the first preferred embodiment. Similarly, the device (400) of the fourth preferred embodiment may look similar the device (200) of the second preferred embodiment of the invention. When the external force is released, the device (300) of the third preferred embodiment will tend to return to its initial non-bent state as shown in FIG. 3 and the device (400) of the fourth preferred embodiment will tend to return to its initial non-bent state as shown in FIG. 4. This elasticity acts to help keep the device in position, by applying a force against the intestinal wall.

### Fifth Preferred Embodiment

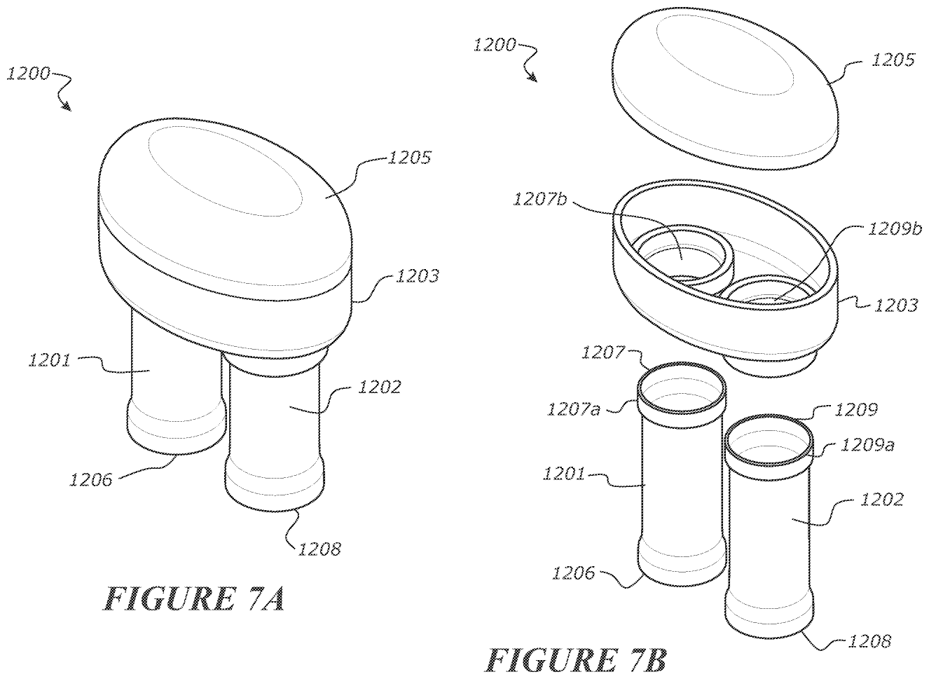

FIGS. 7a-7b show a connecting device (1200) of the fifth preferred embodiment of the invention.

The connecting device (1200) of the fifth preferred embodiment of the invention offers an alternative to the connecting devices (100, 200, 300 and 400) described above.

The connecting device (1200) in this preferred embodiment comprises two tubular members, namely the first tubular member (1201) and a second tubular member (1202), and a hollow body member/intermediate member (1203).

The first tubular member (1201) comprises a longitudinally opposed first and second open ends (1206 and 1207). Similarly, the second tubular member also comprises a longitudinally opposed first and second open ends (1208 and 1209).

The tubular members (1201 and 1202) are preferably made out of a material that is flexible and elastic and therefore has a shape memory and/or resilience, i.e. the tubular member has a property to recover to its original relaxed shape.

The first open ends (1206 and 1208) of the tubular members (1201 and 1202) are preferably flared/slight funnel and therefore the external diameter of each of those flared ends are larger than the internal diameter of the body portion that is not flared. The flaring of the first open end (1206) and second open end (1208) assist in anchoring of the connecting device (1200) when the device (1200) is inserted inside ostomies or stomas on the body of a patient. Such flaring also serve in sealing the inner wall of the orifice or lumen so that leaks of fluid or intestinal content do not occur around the edges of the connecting device (1200).

As shown in FIG. 7b, the second open ends (1207 and 1209) of the tubular members (1201 and 1202) preferably have a collar or flared portions (1207a and 1209a).

The hollow body member (1203) is in a form of a connecting structure as shown in FIG. 7b. The connecting structure comprises two formations, namely an inlet (1207b) and an outlet (1209b) as shown in FIG. 7b. The second open end (1207) and/or the collar or flared portion (1207a) of the first tubular member (1201) is adapted to be secured (coupled/inserted) with the inlet (1207b) and the second open end (1209) and/or the collar or flared portion (1209b) of the second tubular member (1202) is adapted to be secured (coupled/inserted) with the outlet (1209b). For example, at least the second open ends (1207 and 1209) and/or the collar or flared portions (1207a and 1209a) may be collapsed for insertion via the inlet (1207b) and outlet (1209b) and once inserted, they may expand to their initial relaxed or non-collapsed state for retention to the respective inlet (1207b) and outlet (1209b). At least for this reason, it is preferred that the tubular member (1201 and 1202) is made out of material(s) with some ability to collapse for insertion then expand into their relaxed, i.e. initial non-collapsed position for retention. The suitable material(s) may be the same material(s) as described above for the construction the first and third embodiments (100 and 300), or second and fourth (200 and 400) embodiments as described above.

The tubular members (1201 and 1202) may be of mesh structure similar to the ones described above for first and third embodiments (100 and 300) or non-mesh structure similar to the ones described above for the second and fourth embodiments (200 and 400). Alternatively, one of the tubular members (1201 or 1202) may be of a mesh structure and the other tubular member (1201 or 1202) may be of a non-mesh structure.

The collar or flared portions (1207a and 1209a) assist in anchoring of the tubular members (1201 and 1202) when the tubular members (1201 and 1202) are inserted/coupled though the inlet (1207b) and outlet (1209b). Such flaring also serve in sealing the inner wall of the inlet (1207b) and the outlet (1209b) so that leaks of fluid or intestinal content do not occur around the edges of the tubular members (1201 and 1202). Therefore, the collar or flared portions (1207 and 1209a) are preferred. However, any other suitable alternative means for attaching the tubular members (1201 and 1202) to the inlet (1207b) and outlet (1209b) are possible, for example, attachments via threaded connection, clip or snap fit connection, attachment using fasteners, magnetic connection etc.

Alternatively still, the tubular members (1201, 1202) may be integral with the bridging hollow body member 1203.

The intermediate member (1203) is preferably made out of same material as the tubular member, or from different materials. Preferably, the hollow body member (1203) or at least part of it is of mesh structure similar to the ones described above for first and third embodiments (100 and 300) or non-mesh structure similar to the ones described above for the second and fourth embodiments (200 and 400). When applied, the intermediate member also preferably covers and therefore protects the stoma orifices from damage or abrasion.

Optionally the device (1200) comprises a cap (1205) that is a removable.

In some preferred designs, the cap is a magnetic cap. Preferably, the part of the intermediate member (1203) that engages with the cap (1205) is also made out of a magnetic material so that the cap (1205) can be magnetically attached and detached from the intermediate member (1203). However, other alternative means for the engagement of the cap (1205) to the intermediate member (1203) is equally possible such as but not limited to engagement via hinged connection, threaded connection, clipping mechanism, biasing means, fasteners or a combination thereof. The cap (1205) or at least a portion of the cap (1205) may even be of bung-type as described above with reference to FIGS. 2b-2d.

Having a cap (1205) allows access to the inside portion of the hollow body member (1203) as well as of the tubular member(s). Having such access to the inside portions can assist in cleaning up of the inside portions and releasing or flushing of any blockages or potential blockages from the inside portion of the device (1200) when the device (1200) is implanted in the body of the patient.

In a less complex design where caps (1205) is/are not present, it is still possible to flush the contents causing blockages or potential blockages via alternative techniques. For example, a patient may be given a per-oral solution as (such oral ‘gastrografin’ or polyethylene glycol) which can dilute and flush impacted gastro-intestinal blockages.

Alternatively, it is also possible to remove and replace the device (100, 200) altogether in the event of blockages or potential blockages. Alternatively, squeezing by applying external force to the device (1200) from outside, or vibration may also assist in clearing up of any blockages or potential blockages. Optionally, the lining of the device (1200) may be coated with a lubricious material or lubricious liner to aid passage of the contents and to reduce the chance of blockages. For example, polytetrafluoroethylene (or PTFE) or suitable nanoparticles or biocompatible materials may be used as a lubricious material.

Although, not shown, the device may comprise at least one attachment means similar to the attachment means shown in FIG. 2b on the external surface of the first tubular member, second tubular member and/or intermediate member. The attachment member can be in the form of hoop or a loop and is suitable to facilitate attachment to an ancillary device (such as stoma belt).

### Preferred Method of Use

One preferred method of using a connecting device of the present invention will now be described with reference to FIGS. 5a and 5b.

FIG. 5a shows one method of using the connecting device (100) of the first preferred embodiment of the present invention. Specifically, FIG. 5a shows connecting device (100) connecting a first orifice or opening (501) with a second orifice or opening (502). Each of the openings (501, 502) in this example is an ostomy or stoma of a gastrointestinal tract that is typically a surgically created opening.

In an example shown in FIG. 5b, the connecting device (100) comprises optional attachment means (115) in the form of hooks and optional protrusions (116) extending circumferentially above flared portions of both the open ends (106 and 108). The connecting device (100) in this example extends to about 140 mm when measured from the first open end (106) to the second open end (108) with the internal diameter of the non-flared part of the body portion (102) being about 18 mm diameter and the internal diameter of the flared part of the body portion (102) being about 24 mm. It is to be noted that these dimensions are just examples. The diameter and size of the connecting device (100) may be smaller or larger depending upon several factors such as the degree of ‘pout’ of the stoma(s) and/or other anatomical variations e.g. patient size, age, sex, or body mass index or obesity.

At least the flared portion of the first open end (106) of the device (100) is inserted into the first opening (501) of the gastrointestinal tract and at least the flared portion of the second open end (108) of the device (100) is inserted into the second opening of the gastrointestinal tract as shown in FIG. 5a. In this example, about 40 mm the device (100) is inserted into the first opening (501) and about another 40 mm of the device (100) is inserted into the second opening (502). The curved portion forming part of the remaining about 60 mm of the device (100) is not inserted and is therefore exposed outside the skin of a patient. It is to be noted that the insertion and connection lengths given in this example may vary.

The device may be first inserted in a collapsed or compressed form and once inserted, the device (100) expands or tends to expand toward its initial relaxed (non-collapsed state) state. The degree of relaxation achieved is typically limited by tension created by the wall of the bowel.

The insertion is done by using hands and/or by using a suitable insertion tool.

Once, inserted as shown in FIG. 5a, the connecting device (100) provides a flow pathway for the digestive content to flow from the first opening (501) to the second opening (502).

The flared open ends (106 and 108) abut against the wall of the gastrointestinal tract and therefore allow the device (100) to be anchored thereby preventing accidental removal. Such flaring also serves in sealing the inner wall of the orifice or lumen so that leaks of fluid or intestinal content do not occur around the edges of the connecting device (100). The optional protrusions (116) provide further grip by abutting against the gastrointestinal tract thereby further facilitating the anchoring of the device (100).

The connecting device (200) of the second preferred embodiment of the invention as shown in FIGS. 2a-2d may be used in the same way as described above.

With regards to the connecting devices (300 and 400) according to the third and fourth preferred embodiments described above, the devices (300 and 400) may be used in a similar manner except they are first bent in an arcuate shape such as U-shape or V-shape or substantially U-shape or V-shape.

Connecting devices (100, 200) that are curved shaped during relaxed state as shown in FIGS. 1 and 2 are advantageous as the angle of such curved devices (100, 200) are less acute in the relaxed state. This means, when inserted, such connecting devices expand outwards slightly against the walls of the gastrointestinal tract (or small or large intestine). This is shown in FIG. 5b. Such slight expansion outwards against the walls of the gastrointestinal tract is advantageous as it enough to aid retention of the devices (100 and 200) during peristalsis of the bowel, but at the same time not so much that the pressure on the bowel causes local loss of blood supply or “pressure necrosis”). At least for that reason, connecting devices (100, 200) of the first and second preferred embodiments may be considered to be more desirable over the connecting devices (300, 400) of the third and fourth preferred embodiments of the present invention.

It will be appreciated that the amount of bending deformation, and the elastic properties of the devices (100, 200, 300, 400) will determine how much spring back force is applied to the intestinal wall. It is intended that these properties may be tailored to suit the desired circumstances.

One preferred method of using a connecting device (1200) of the fifth preferred embodiment will now be described.

At least the flared portion of the first open end (1206) of the first tubular member (1201) is inserted into the first opening (501) of the gastrointestinal tract and at least the flared portion of the first open end (1208) of the second tubular member (1202) is inserted into the second opening (502) of the gastrointestinal tract.

The tubular ends (1201 and 1202) are first inserted in a collapsed or compressed form and once inserted, the tubular ends (1201 and 1202) expand or tend to expand to their relaxed (non-collapsed state) state.

Such insertion is done by using hands and/or by using a suitable insertion tool.

At least the second open end (1207) of the first tubular member (1201) is coupled/inserted to the inlet (1207b) of the intermediate member (1203) and at least the second open end (1209) of the second tubular member (1202) is coupled/inserted to the outlet (1209b) of the intermediate member (1203). The intermediate member (1203) forming part of the device (1200) is not inserted and is therefore exposed outside the skin of a patient.

If the cap (1205) is present, it is ensured that the cap is closed.

In that way, the connecting device (1200) provides a flow pathway for the digestive content to flow from the first opening (501) to the second opening (502).

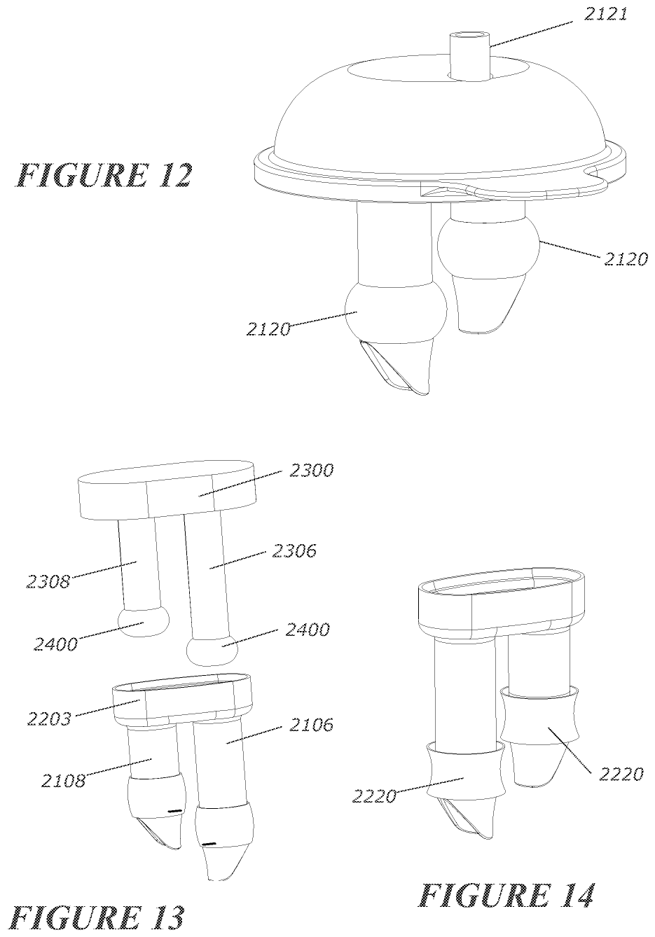

When the first tubular member (1201), the second tubular member (1202) and the intermediate member (1203) are assembled together as shown in FIGS. 12a and 12b, the first open end (1206) of the first tubular member (1201) becomes the first open end of the connection device (1200) and first open end (1208) of the second tubular member (1202) becomes the second open end of the connection device (1200).

As it can be seen, the connecting devices (100, 200, 300, 400 and 1200) of the preferred embodiments of the present invention are simple to use, and unlike traditional devices, they do not require a traditional stoma bag.

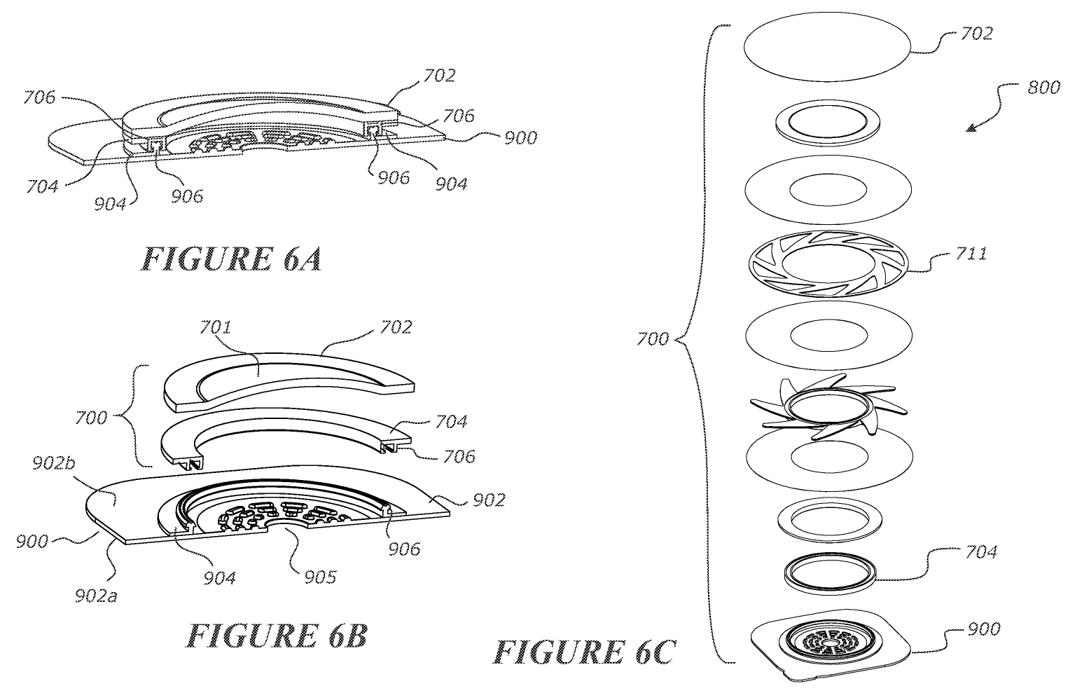

FIGS. 6a-b show a pad according to one preferred embodiment of the present invention. The pad (702) is made out of a fluid resistant or partly fluid resistant material that is adapted to cover at least one orifice formed on a patient's body during surgery. The orifice may be an ostomy or stoma of a gastrointestinal tract that is a surgically created opening.

Preferably, the fluid resistant material is neoprene or a rubber or a latex, or silicone or suitable polymer.

The pad (702) is preferably circular or substantially circular in shape.

As shown in FIGS. 6a and 6b, the pad may optionally comprise a convex structure (701).

Although not shown, the pad may comprise multiple layers that are connected to each other or are integrally formed at peripheral edge portions. One of the multiple layers may optionally be a biasing member (such as a spring) that is adapted to increase the flexibility and/or resilience of the pad. The biasing member may also be in a form of a lattice.

In use, the pad (702) may function as a covering device. In other words, the pad (702) may be used to cover or seal the ostomy or stoma and an adhesive tape may be used on top the plate and onto the skin to prevent the pad (702) from being detached from the skin.

The pad (702) may optionally comprise straps to enable the user to wear it around the waist and/or attachment means to connection to the ancillary devices (such as stoma belt). Alternatively, the pad (702) may be formed as a part of a stoma belt, such as the belt made of neoprene and/or hook and loop fasteners.

As described above, the pad (702) may function as a covering device.

FIG. 6b shows an exploded view of a covering device (700) according to a further preferred embodiment of the present invention. The covering device (700) is suitable for covering at least one orifice formed on a patient's body during surgery. The orifice may be an ostomy or stoma of a gastrointestinal tract that is a surgically created opening.

The covering device (700) is a two-piece device. The first piece is the pad (702) which is substantially the same as the pad (600) as described above. The second piece is a coupling member (704) that is adapted to be supported by the pad (702). The coupling member (704) comprises a coupling structure (706) that is configured to connect with a cooperating coupling structure of another coupling member that is either a part of the covering device (700) or is external to the covering device (700).

The coupling member (704) is preferably a clip in a form of a ring.

The coupling structure may be in the form of a groove adapted to secure a cooperating protrusion(s). Although, not shown, the coupling structure of the coupling member (704) may in the form of a protrusion adapted to be secured in a cooperating groove(s).

Although, not shown, the covering device (700) may optionally further comprise a biasing member (such as spring) that is either located or is adapted to be located between the pad and the first coupling member in order to increase the flexibility and/or resilience of the pad. The biasing member in one preferred form may be in a form of a lattice.

Although, not shown, the covering device may optionally comprise at least one attachment means connected to the membrane. The attachments means (such as hook or a loop) that may be suitable to facilitate attachment to an ancillary device.

### Plate Structure

FIG. 6a shows a top perspective view of a plate structure (900) according to one preferred embodiment of the present invention. FIG. 6b shows a bottom perspective view of a plate structure (900) of FIG. 6a. The plate structure (900) is configured to be removably attached to a skin of a patient and around at least one orifice formed on a patient's body during surgery. The orifice may be an ostomy or stoma of a gastrointestinal tract that is a surgically created opening.

The plate structure (900) comprises a thin membrane (902) having a first side (902a) and a second side (902b). The first side (902a) of the membrane (902) is configured to be attached to a skin of a patient using an adhesive. There is a coupling member (904) that is configured to be attached to the second side (902b) of the membrane (902). The coupling member (904) comprises a coupling structure (906) that is configured to connect with a cooperating coupling structure of another coupling member that is either a part of the plate structure (900) or is external to the plate structure (900).

The coupling member (904) is preferably a clip in a form of a ring as shown in FIGS. 6a & 6b.

The membrane (902) is preferably made up of a plastic or vinyl material. Preferably, the first side (902a) of the membrane (902) has an adhesive coating to adhere to (and preferably seal with) the skin of a patient. The adhesive coating is preferably covered by a suitable protective layer which is adapted to be peeled out to during use to reveal the adhesive coating. Alternatively, instead of having adhesive coating, the adhesive may be manually applied to one side of the membrane at the time of use.

The coupling structure (906) of the coupling member (904) may be in the form of a groove adapted to secure a cooperating protrusion(s). Alternatively, the coupling structure (906) of the coupling member (902) may in the form of a protrusion adapted to be secured in a cooperating groove(s).

The membrane comprises an aperture (905) at or near the centre of the membrane (902) and the size of the aperture is substantially the same or substantially similar to the size of the orifice formed in the patient's body over which the plate structure (900) is intended to be used. Optionally, the plate structure (900) does not have a preformed aperture(s) (905), in which case user actually makes the aperture(s) of suitable size by using a cutting tool, prior to use. The membrane (902) may even have perforations to facilitate creating the aperture prior to the use of the plate structure.

Although, not shown, the plate structure (900) may optionally comprise at least one attachment means connected to the membrane. The attachments means (such as hook or a loop) may be suitable to facilitate attachment to an ancillary device.

In use, the protective covering material is peeled out by the user thereby exposing the adhesive coating. In an embodiment, where no adhesive coating is already present in the membrane, the user manually applies adhesive on first side (902a) of the membrane. The aperture(s) (905) that is/are either already present in the membrane (902) or is created in the membrane (902) is/are then aligned with the orifice(s) formed on the patient's body to ensure that the orifice(s) is/are not covered with the membrane (902) and the first side (902a) of the membrane (902) is then attached to the skin of the patient.

FIGS. 6a-c, shows how the plate structure (900) and the covering device (700) are secured together during use. As shown, the coupling member (704) of the covering plate (700) is configured to be secured to a coupling member (904) of the plate structure (900). More specifically, in this particular example, the coupling structure (706) of the coupling member (704) of the covering device (700) is formed as groove. That groove is adapted to seal or secure the coupling structure (906) of the coupling member (904) of the plate structure (900) that is formed as a protrusion. One type of seal or secure would be a snap fit connection.

In one option, the apparatus shown in FIGS. 6a & 6b comprising the plate structure (900) and covering device (700) is manufactured and supplied as a kit of parts for construction of a cover that is suitable for covering at least one orifice formed on a patient's body during surgery.

FIG. 6c shows a kit (800) of parts comprising at least a plate structure (1115), and a covering device (700) comprising a pad (702), at least one biasing member (711), and a coupling member (704) together with other optional components as shown. The biasing member(s) (711) provides support to the connecting device. The biasing member (711) is in the form of lattice.

When the pad (702) is pushed outwards, the outer edge of the biasing member (711) lifts out, providing the spring effect. Then a stiffer material can be used for the pad (702).

A connection device may be in a form of a kit comprising: a connecting device (100, 200, 300, 400 or 1200) as described first, second, third, fourth or fifth aspects, and a pad (702) as described above. In use the pad (702) is adapted to be supported on a portion of the connection device that would otherwise be exposed outside a body of the patient.

The kit may further comprise a plate structure (900) as described above. In use, the coupling member (704) of the covering device (700) is adapted to be coupled to coupling member (904) of the plate structure (900).

### Further Preferred Method of Use

Once the connecting device of the present invention has been implanted such as in a way as described above with reference to FIGS. 5a and 5b, an adhesive material may simply be attached to secure the portion that of connecting device (100, 200, 300, 400, 1200 or 2100) that is exposed outside the skin of the patient.

One preferred method for using the connecting device (100, 200, 300, 400, 1200 or 2100) together with the one or more of other devices as described above in order to connect a first orifice with the second orifice will now be described. Each orifice in this example is an ostomy or stoma of a gastrointestinal tract that is typically a surgically created opening. The method comprising at least the following steps:
positioning and attaching the first side (902a) of the membrane (902) of the plate structure (900) to the skin of a patient; and
inserting the first open end (106, 206, 306, 406 or 1206) of the connecting device (100, 200, 300, 400 or 1200) to the first orifice and the second open end (108, 208, 308, 408 or 1208) of the connecting device to the second orifice.

The first open end (106, 206, 306, 406 or 1206) and second open end (108, 208, 308, 408 or 1208) are inserted to the respective orifice through at least one aperture that is either already present in the membrane (902) or is created in the membrane (902).

The method may further comprises positioning an adhesive material over any portion of the connecting device that is external to the patient's body. Alternatively or additionally, the method further comprises a step of positioning a pad (600) as described above over any portion of the connecting device (100, 200, 300, 400 or 1200) that is external to the patient's body. Alternatively or additionally, the method further comprises the step of coupling the coupling member (904) of the plate structure (900) with the coupling member (704) of the covering device (700).

### Integrated Devices

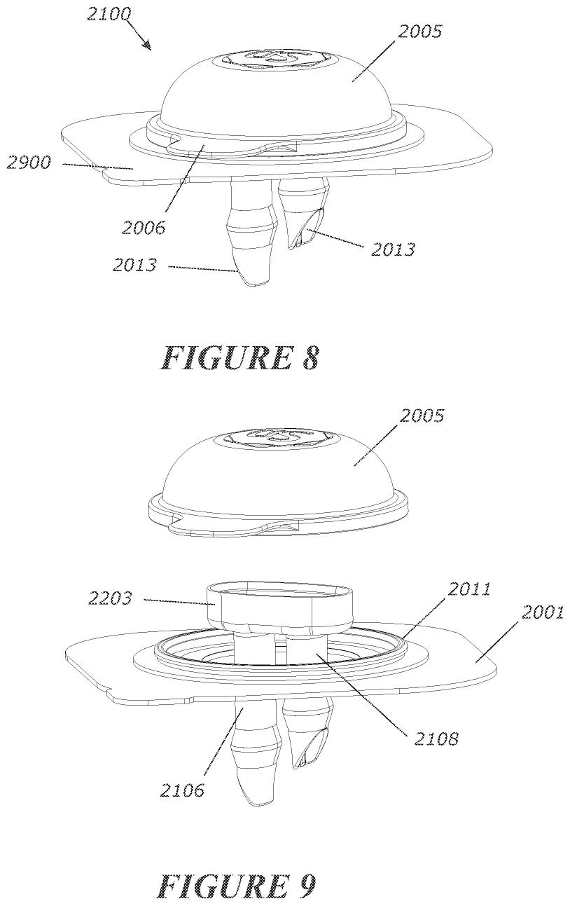

With reference to FIGS. 8 to 18B, various additional features of the device 1200 will now be described. It will be appreciated that one or more of the individual features described in these figures may be incorporated in combination to achieve different alternative embodiments.

With particular reference to FIGS. 8 and 9, an integrated device 2100 is shown the integrated device 2100 comprises a hollow body member 2203, and first and second tubular legs 2108, 2106 in fluid communication with, and dependent from the body inlet & outlet. The hollow body member 2203 includes a body opening, and a cap 2005 closes the body opening to complete the pathway between the legs.

It is anticipated that all described configurations of the device may be adapted for different patients with different needs. For example, the components of the device should be sized in profiled as required for neonatal patients or adult patients.

In some configurations, tubular members are between 8 French and 12 French in size, adapted for neonatal patients.

In other configurations, the tubular members are between 32 French and 42 French in size, adapted for adult patients.

In some configurations a flexible plate structure 2900 with an adhesive backing in order to adhere to, and seal with a patient's skin is provided as before. In this embodiment a connecting ring 2001 is provided on the flexible plate 2904 directly coupled to the shell 2005.

Preferably, the cap 2005 defines the shell as described in FIGS. 7a & 7b. The shell 2005 substantially encompasses and defines a volume of space surrounding the stomas.

Preferably, the volume of space surrounding said first and second surgically created orifices is isolated from the pathway between the first tubular member and the second tubular member so that contents being transferred from the first to second surgically created orifice does not leak into the surrounding volume of space.

It is preferred that the coupling between 2005, and ring 2001 is substantially leak resistant. In the illustrated embodiment, a ridge 2011, is adapted to couple with a corresponding groove 2012 (as shown in FIG. 11), although it will be appreciated that these features may be reversed, or any other suitable coupling employed.

The coupling allows secure retention of the shell 2005 on flexible plate 2900, while also allowing for a patient/doctor to reasonably easily remove 2005. For this purpose, tab 2006 may be provided to assist.

In other configurations, a plate structure 2900 is absent, and the shell 2005 sits directly against a patient's skin. This may be useful for premature neonates or other patients with fragile skin. The skin of these patients may be damaged or torn by adhesive such as on some traditionally base plates.

In some configurations, the shell 2005 comprises a flat rim 2009 extending from a perimeter 2008 of the shell. The flat rim 2009 having an underside configured to sit against a patient's skin. The flat rim provides a flat surface which can be placed comfortably onto the patient.

It is anticipated that the flat rim 2009 configured to sit against a patient's skin may be integrated with the shell 2005, integrated with another part of the device, or is a separate component coupled to the device.

The device 1 may be attached to the patient by adhering the shell to the patient. Preferably, the underside of the flat rim 2009 comprises an adhesive to adhere the shell to the patient's skin.

In other configurations, the device is not adhered onto the patient. For example, the device may be held in place using a soft strap.

Preferably, the shell 2005 is rigid or semi-rigid and adapted to exert a downward pressure to keep the legs within the surgically created orifices when the device is attached to a patient.

As illustrated best in FIG. 11, cap 2005 also integrally provides cap portion 2205 which when in position completes the pathway between inlet leg 2106, and outlet leg 2108 of the device 2100.

In the preferred configurations, the cap 2005 comprises a pathway seal on an inner surface/underside of the cap to seal the body opening as best shown in FIG. 11.

Optionally, the cap 2005 as referenced in FIG. 9 may be replaced with a cap with an open top to allow draining of digestive contents. The draining cap is similar to the shell 3200 with an aperture 3201 as illustrated in FIG. 20 (to be discussed later).

This cap would be useful in situations such as straight after acute surgery, where the device could be inserted into a swollen stoma to keep it flowing and prevent obstructions. Then when ready, the cap 2005 without an aperture could be positioned over the body 2203 to complete the pathway between inlet leg 2106, and outlet leg 2108 of the device 2100, and allow nutrient recycling from the proximal to the distal stoma.

If as illustrated in FIGS. 8, 9 and 11, the preferred device is reduced to 3 components being, main body 2203, flexible plate 2900 and 2205, then the operation of the device can be simplified. The reduction of parts may have the advantage of less assembly being required, as well as less potential leak pathways.

The embodiment illustrated in FIG. 8 also includes leg ends 2013, on each of the inlet and outlet legs 2106 and 2108 respectively. Each leg end 2013 comprises an aperture for the digestive contents to flow between the device and the intestinal tract.

In some configurations, the leg ends 2013 of the tubular members are bevelled, to define an oblique aperture 2015. It will be appreciated that this feature may be included on other described embodiments.

In the preferred configurations, the bevelled end apertures of the first tubular member and the second tubular member face away from each other.

The bevelled ends 2013 may assist insertion of the device, while maintaining a sufficiently large cross-sectional area of the aperture. The bevelled end 2013 arrangement also helps to prevent a wall of the intestinal tract from flapping over, or covering the inserted ends of the device.

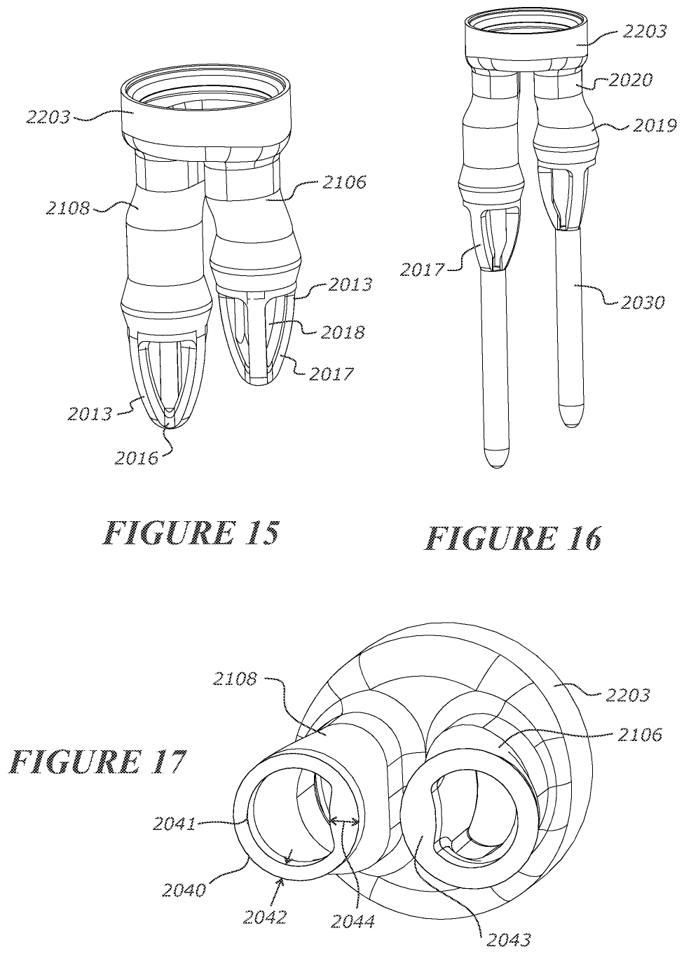

In another configuration, one or both leg ends 2013 comprise a cage feature as shown in FIGS. 15 to 19. Preferably, the cage feature is tapered, so that it reduces in diameter as it extends away from the body 2203 of the device. The cage feature may be tapered linearly or non-linearly.

The tapered-cage leg end forms a nose 2016 (referenced in FIG. 15) at the tapered end, such that it is adapted to aid insertion of the legs into a stoma and into the intestinal tract. A device with this tapered profile may not need a separate introducer element to help insert the device into the stoma.

As the tapered-cage leg end is introduced into the intestinal tract, the intestinal tract is gradually forced open. The profile of the leg end is able to guide the device through the stoma without causing injury to the stoma and/or intestinal wall. The tapered-cage leg may assist insertion of the device, making it easier for a medical professional, or a patient themselves to insert the device into the stoma.

In the preferred configurations, the tapered cage feature comprises cage supports 2017 to form the tapered leg end profile. The tapered cage feature comprises cage openings 2018 formed between neighbouring cage supports, to allow flow of digestive contents to pass. Preferably, the cage openings 2018 are sized and configured so that the digestive contents can easily flow between the cage supports.

Preferably, the cage openings 2018 are big enough so that the cage feature does not unduly inhibit the flow of digestive contents between the device and the intestinal tract.

In the preferred configurations, the cage openings 2018 have a total area of at least the cross-section of the leg it depends from to achieve this.

Preferably, the cage openings are substantially triangular, due to the tapering of the leg ends.

Preferably, the tapered cage feature is formed by two or more cage supports 2017. In the illustrated configuration, the tapered cage feature is formed by three cage supports.

The cage supports keep the intestinal tract open and prevent the intestinal wall from collapsing by engaging and holding the internal walls of the intestinal tract apart.

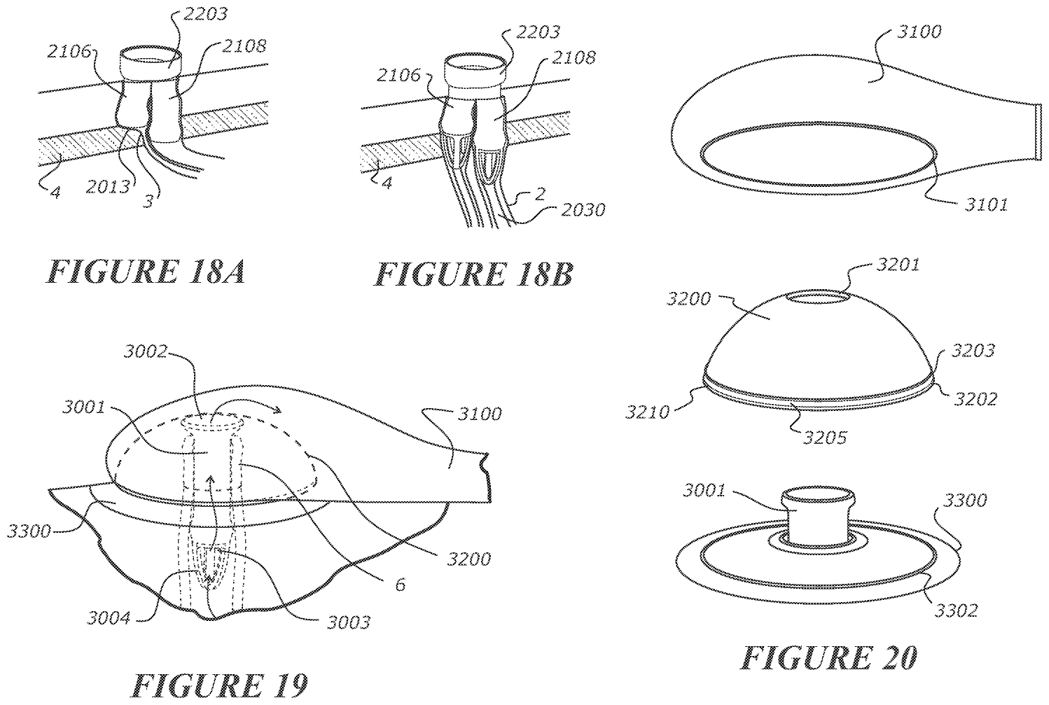

As the cage supports 2017 press against the internal walls 2, they prevent the intestinal tract wall 2 from collapsing over the leg end as shown in FIG. 18b. This feature may be particularly useful when there is a low/no flow of digestive contents.

As shown in FIG. 18A, a wall 3 of the intestinal tract may form a flap and impede flow through an end of the leg. An obstruction may occur as the walls of the intestinal tract are forced together at denser facia 4 or muscle tissues. The cage feature as shown in FIG. 18B can help prevent this from occurring.

Keeping the intestinal tract open is important to provide a minimally restricted open pathway for the digestive contents to travel through. If the intestinal tract is not held open, the intestinal walls tend to collapse or be compressed inwards, reducing the diameter of the intestinal tract. Higher pressures are generated to overcome the reduction in flow area.

High intestinal pressure may cause discomfort to a patient. Furthermore, high pressure in the intestinal tract is not desirable as it can cause injury to the intestinal wall, blockage. It is important to avoid pressure build up as it may cause the patient to develop symptoms such as bloating, nausea, pain or vomiting. Furthermore, a build-up of pressure is not desirable as the digestive contents may rush into the device, increasing the likelihood of leaks.

Preferably, the digestive contents flow from an inlet leg 2106 adapted for a proximal intestinal opening to an outlet leg 2108 adapted for a distal intestinal opening. As this device is passive in the preferred configurations, the digestive contents only flow out of the leg adapted for the distal intestinal opening if enough pressure is generated to urge the digestive contents from the proximal leg to the distal leg.

Preferably, the cage supports are equally distributed round a longitudinal axis of the cage feature to hold the intestinal tract open.

Optionally, 3 cage supports are located around the tapered cage end and spaced 120° apart.

Optionally, 4 cage supports are located around the tapered cage end and spaced 90° apart.

The structure of the tapered cage end is a multi-purpose feature which provides a synergy between ease of insertion, keeping the intestinal tract open to allow the device to work effectively, and maximising the area of the openings to allowing digestive contents to flow through.

Preferably, the structure of the tapered cage end allows the device to be inserted into a patient without the need of medical equipment or a dedicated introducer device, improving the ease of insertion. This is advantageous as a person without the tools or medical training may be able to easily insert the device.

Optionally, the legs of the device further comprise longitudinal guide members 2030 as shown in FIGS. 16 & 18B. The longitudinal guide members 2030 extend from a distal end of the legs. Preferably, the longitudinal guide members 2030 comprise a diameter less than the other parts of the device which are adapted to be inserted into the stoma.

In the preferred configurations, the longitudinal guide members 2030 have a diameter between 5 and 12 mm.

In the preferred configuration, the longitudinal guide members 2030 have a length between 20 and 60 mm.

Longitudinal guide members 2030 aid with insertion of the device through a stoma and into the intestinal tract 2 by providing a length of the device that can easily pass into the stoma prior the portions of the device adapted to the inserted into the stoma following the longitudinal guide member. The longitudinal guide members 2030 having a smaller diameter may be inserted through the stoma with less resistance than the subsequent portions of the device.

In the preferred configurations, the longitudinal guide members 2030 are flexible. Flexible longitudinal guide members 2030 may allow easy insertion through the stoma and into the intestinal tract 2 with reduced discomfort, as the rods can gently straighten the intestinal tract while allowing for the natural profile/bends of the intestinal tract as shown in FIG. 18B.

Preferably, the longitudinal guide members 2030 comprises a compliant and flexible material such as silicone or other suitable materials known to a person skilled in the art.

The longitudinal guide members 2030 as described improves the ease of insertion of the device. This is advantageous as it can give a patient confidence to insert the device into the right position.

Furthermore, flexible guide members 2030 can help flex to the shape of the intestinal tract, to pass into the lumen and/or gently straighten the intestinal tract at or near the inlet and outlet of the device.

In the preferred configurations, the external wall of the inlet and outlet legs forms a circular cross-section profile adapted to be inserted into a stoma and intestinal tract. A circular leg profile allows the legs to be inserted with minimal discomfort and reduces the likelihood of injury to the stoma and intestinal wall.

In some configurations, an external sidewall 2040 of the inlet and outlet legs forms a circular profile, such that the wall thickness 2042 of the legs are uniform around the perimeter of the legs.

In other configurations, a sidewall of the inlet and outlet legs forms a non-circular profile, such that the wall thickness 2042 of the legs are not uniform around the perimeter of the legs as shown in FIG. 17.

In one configuration, the legs comprise an internal rib 2044 which protrudes from a sidewall of the leg, so that the internal wall 2041 of the leg protrudes inwards. The internal rib increases the wall thickness in a thickened wall region of the leg to increase the stiffness of the leg without reducing the flow path area too much.

By increasing the stiffness of the leg, the internal rib 2044 helps with insertion of the leg, by keeping the leg relatively rigid when the leg is formed from a flexible material. A stiffer leg with an internal rib 2044 can reduce the likelihood of the leg collapsing, such as when the leg telescopes due to the pressure exerted at the leg end during insertion.

A leg with an internal rib 2044 provides a leg with a stiffer structure, which can maintain its structure in a longitudinal direction so that the device is more likely to be inserted completely through the stoma and to lie in the correct open position. This feature may improve the ease of use of the device and the likelihood of a patient being able to insert the device successfully themselves.

The internal rib extends along the longitudinal axis of the leg. Preferably, the internal rib extends along a substantial length of the leg.

In some configurations, the inlet leg 2106 and outlet leg 2108 of the device have different lengths as best shown in FIGS. 9 and 15.

In the preferred configurations the inlet leg 2106 adapted to be inserted into the proximal end is shorter than the outlet leg 2108 adapted for the distal end. It may be desirable for the leg adapted to be inserted into the distal end to be longer to direct the digestive contents further downstream in the intestinal tract.

The different leg lengths correspond with a typical orientation of a stoma. The inlet leg 2106 is shorter so that the device sits better in the stoma. This is because generally the proximal limb of stomas are everted so that the digestive contents are directed outwards external to the patient body (and into a stoma bag). The everted stoma helps ensure the digestive contents are directed away from a patient's skin and to reduce the likelihood of leaks.

In effect, a shorter inlet leg 2106 should result in both ends of the device 2013 to be situated at similar depths within the intestinal tract.

Furthermore, off-setting the inlet and outlet legs 2106, 2108 may be desirable as it may mean protruding or flared regions (e.g. cuffs 2120) of the legs are not located adjacent each other. Off-setting the legs will limit the likelihood of generation of excessive pressure as the flared regions in the respective legs push against each other. Excessive pressure could lead to a loss of blood supply and pressure necrosis, causing perforation into the abdominal wall, which would be a severe complication.

In other configurations, the inlet leg 2106 is longer than the outlet leg 2108.

In some configurations, the inlet leg comprises a diameter great than the outlet leg. A narrower outlet leg may be use useful for cases of narrow distal openings, e.g. in adults with distal venting stomas or mucous fistula openings or stenotic distal openings due to disuse atrophy or ischaemia, and also seen in children after gut atresia surgery.

Optionally, the device comprises absorbent material, such as gauze, foam, wax, sponge, or a gel (not shown), in the space 2014 under 2005 (referenced in FIG. 11), in order to achieve a more effective seal, and to capture and/or limit any leakage that may occur around the legs of the device.

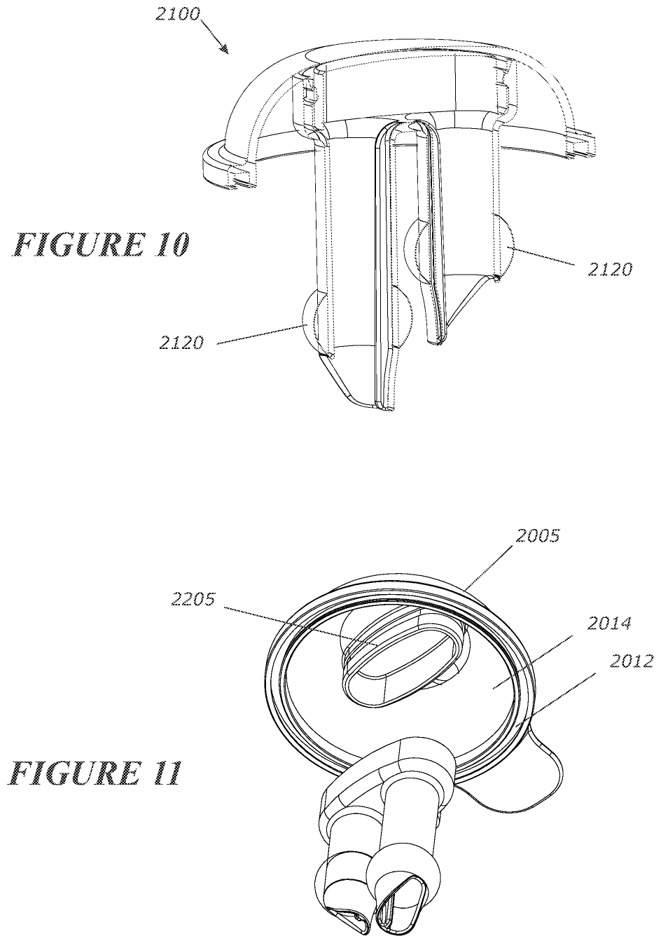

With particular reference to FIGS. 10 and 12, further optional features of device 2100 will be described. In these embodiments, cuffs 2120 are configured as inflatable ring balloons located at the ends of the legs of the device 2100.

In these embodiments, after the device 2100 is introduced into a stoma, each cuff 2120 can be inflated via injection of saline or other suitable gas/liquid. For this purpose inlet 2121 is provided and is in fluid communication with each cuff 2120. A valve may be provided (not shown), in order to control the fluid flow to and/or from each cuff. It will be appreciated that may have improved sealing in the bowel, and/or improved retention.

With reference to FIG. 13, an introducer apparatus 2300 suitable for assisting in the positioning of a connecting device 2100 into a stoma is illustrated. The device is preferably rigid, and takes a form suitable for insertion into the connecting device 2100. The introducer apparatus 2300 includes leg portion 2308 and leg portion 2306 shaped and oriented to fit inside connecting device leg 2108 and 2106 respectively. With the introducer 2300 inside the resilient and flexible connecting device 2100, the connecting device can be more easily inserted into a stoma.

Introducer 2300 may optionally include bulbous regions 2400, at or towards each end in order to assist with the deployment of an appropriate retaining cuff, as described below.

Alternatively, an introducer capable of introducing the legs sequentially may be preferred.

With reference to FIG. 14, a further alternative retaining cuff 2220 as illustrated. In this embodiment, retaining cuffs 2220 have a natural relaxed state which is concave outwardly as illustrated in FIG. 14. In this embodiment, the retaining cuffs 2220 are also of a flexible and resilient material having some shape memory.

The cuffs 2220 take up a convex outward shape when an introducer 2300 (as shown in FIG. 13 and including bulbous regions 2400), is inserted into the connector body 2203. In this state, the introducer and connector body can be inserted into a stoma. Upon withdrawal of the introducer 2300, the bulbous regions 2400 no longer urged the cuffs 2220 into a convex outward condition, and the cuffs 2220 relax into their natural concave outwardly condition. This effectively causes the retaining cuffs to ‘invert’ thereby creating a vacuum against the bowel wall, which may aid in sealing and/or retention.

In further alternative designs, the legs of the device may also take a semi-circular or oval shape, in order to optimally pass and align through the circular stoma aperture of the skin.

### Single-Leg

In another configuration, the device comprises a single tubular leg member 3001 as shown in FIG. 19. The single tubular leg member is flexible and resilient.

It is anticipated that any of the features described for the double legged configurations may be included in the single-legged configurations.

A single tubular leg device in some configurations is adapted to be inserted into an end stoma. The one-legged device may also be useful in urostomies, where an end-stoma is created from a segment of the small bowel in order to create a urine reservoir after a bladder resection.

Alternatively, the single tubular leg device may be inserted into a proximal stoma. This may be particular useful to receive digestive contents from the stoma.

The single-legged device typically acts as a stoma adaptor which may be used to direct digestive contents out of a stoma away from a patient's skin and out into a stoma bag 3100 for example as shown in FIG. 19.

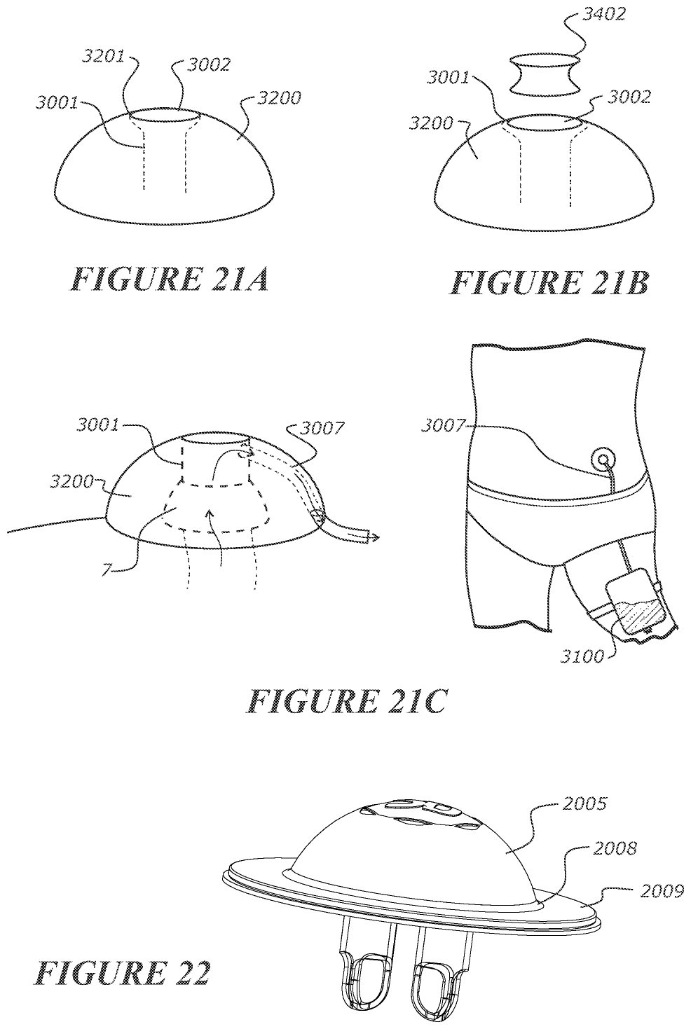

In periods of non-use the device connected to the stoma may be blocked using a cap or plug 3402 as shown in FIG. 21B such as when a patient is undertaking exercise for example. Preferably the cap or plug 3402 closes the shell aperture.

The features described above in the other configurations may be included in the single tubular leg configuration, to provide similar advantages of a simple passive device, which may be easily inserted, stays within a stoma, and is less prone to leaks.

In some configurations, the single leg 3001 device is similar to the described configurations in FIGS. 7A to 18B, but with one tubular leg member. The device according to this single leg configuration comprises a body opening 3002 and an inlet 3003 located at a distal end away from the body opening, the inlet adapted to be inserted into the surgically created orifice.

In the preferred configurations, the single-legged device comprises a cage feature 3004 having cage supports and cage openings between neighbouring cage supports. The cage feature as previous described in the two-legged connecting device provides the same advantages in the single-leg device.

In the preferred configurations, the single-legged device comprises a plate structure 3300 as illustrated in FIG. 20. The plate structure is similar to the structure 2900 described in the configuration described and shown in FIGS. 8 and 9.

In some configurations, the single-legged device comprises a shell, placed against a patient's skin, with a flat rim extending from a perimeter of the shell like the structure as described and shown in FIG. 22.

In one configuration, the single leg device comprises a cap for closing said body opening forming an inner chamber, similar to the cap 1205 shown in FIG. 7B.

In the preferred configurations, the single-legged device comprises a shell 3200 that substantially encompasses and defines a volume of space surrounding the stoma, such that the volume of space is isolated from the inner chamber and the contents entering the tubular member do not leak into the surrounding volume of space.

In another configuration as shown in FIGS. 19 and 20. The shell 3200 substantially encompasses and defines a volume of space surrounding the stoma. The shell 3200 has an internal and external side that are opposite one another.

In some configurations, the shell 3200 comprises an aperture 3201. The aperture 3201 allows digestive contents to flow out of the tubular leg 3001, as indicated by the arrows in FIGS. 19 and 21A. The aperture 3201 may be temporarily blocked using a plug 3402, as shown in FIG. 21b.

Preferably, the shell 3200 is connected to the leg/tubular member 3001 at or towards the body opening 3002 so that digestive contents exit the body opening on the external side of the shell (FIG. 19). The interaction of components provides a clean flow path for digestive contents to flow out of the stoma and away from the skin (and plate structure), and to avoid leaks. Preferably, the internal side of the shell 3200 is isolated from the digestive contents and remains ‘clean’.

The shell member 3200 preferably provides an isolated chamber between a patient's skin (or a plate structure), and the containment of digestive contents in a stoma bag.

The shell member 3200 preferably comprises a shell coupling member 3202 as shown in FIG. 20. The shell coupling member is configured to be connected to a cooperating coupling member.

Preferably, a first shell coupling member 3202 is connected to a plate coupling member 3302. Connection of the shell and the plate coupling keeps the shell on the plate member. As the shell is anchored to the plate, a downward pressure is applied to the tubular leg 3001 so that it remains inserted in the stoma.

In the preferred configurations, a stoma bag 3100 is adapted to cover the body opening 3002 to receive digestive contents from the body opening. Preferably, the stoma bag 3100 comprises a bag coupling member 3101 to adapted to connect the bag to the shell 3200.

Preferably, the shell comprises a second coupling member 3203. The bag coupling member 3101 is adapted to be connected to the second shell coupling member 3203.

Preferably, the shell coupling members run around the perimeter of the shell, to provide a watertight coupling with the other components of the device.

In some configurations, the bag 3100 is coupled to a lower perimeter 3210 of the shell. Coupling at the lower perimeter allows the bag to sit over the entire shell to provide a compact device as shown in FIG. 19.

In some configurations, the first shell coupling member 3202 and the second shell coupling member 3203 have substantially the same diameter.

In the preferred configurations, the first shell coupling member 3202 and the second shell coupling member 3203 is a double-sided, integrated coupling structure 3205. An integrated coupling structure provides a simple structure for the shell to connect both the plate structure 3300 and the bag 3100.

The integrated coupling structure 3205 in some configurations, provides an external or upward facing coupling to interface with the stoma bag 3100, and an internal or downward facing coupling to interface with the base plate structure 3300.

In some configurations, the device further comprises a tube 3007. Preferably, the tube 3007 conveys contents away from the stoma (via leg 3001), and to the outside of shell 3200 via a sealed aperture. The tube may thereby carry contents away from the stoma site to a more convenient place to hold a bag, rather than on an abdomen, such as on a leg strap. This may be more convenient and preferred by patients with particularly occupations or if they are performing a particular activity. For example, a tube to lead the digestive contents away to a stoma bag situated somewhere other than the abdomen (e.g. the floor) may be useful to allow a patient to have a more comfortable sleep.

It is anticipated that the described configurations of the device may be used in for different patients with different medical needs.

In some configurations, the device may be adapted to be used for patients with urostomies as shown in FIG. 21C. Urostomies 7 are stomas used to transport urine out of the body after bladder removal (cystectomy), usually in the context of bladder cancer (or cancers invading bladder). The ureters are sewn on to an isolated segment of small bowel that exits the body in the same way as an end-stoma. In this configuration, a tube 3007 is provided to convey the urine away to a more discrete or useful location.

For example, a patient may prefer to carry the weight of a urine bag on their leg rather than their abdomen. Moving the weight of a urine bag away from the stoma seal can also help to reduce tension on the seal and therefore reduces the likelihood of leaks.

### Variations

It is anticipated that while the foregoing has been given by way of illustrative example of the present invention, all such modifications and variations thereto as would be apparent to a person skilled in the art are deemed to fall within the broad scope and ambit of this invention as is hereinbefore described.

In the preferred embodiments, the flared ends of the connecting devices are shown to be present in both the open ends. While that is most desirable, it may be possible that the flared end is only present in one of the two ends. Optionally, the connecting device may be designed so that it contains suitable additional and/or alternative means or ways for anchoring on one of the open ends.

In the preferred embodiments, the connecting devices are shown to be used for connecting ostomies or stomas of a gastrointestinal tract that are surgically created in order to divert digesting contents from one ostomy to another. However, the connecting devices may equally be used or other connecting other suitable orifices (including disease-created orifices) formed on a body of humans or other animals in order to divert the contents from one orifice to another.

In the preferred embodiments of FIGS. 1 to 5b, the body portion of each connecting device is shown to be a single piece. However, the body portion may alternatively be formed of two or more pieces that are then connected together to form one piece body portion. In one example, the body portion may be formed of two separate pieces that are joined at least with a cap, i.e. a removable cap as described above. Other alternative methods of joining or connecting or coupling of multiple pieces could be wielding, gluing, using fasteners etc. Alternatively, each piece may be provided with suitable coupling mechanisms such as but limited to threaded arrangements, clip arrangements, snap fit arrangements and/or arrangements for magnetic connections.

The connecting devices may be formed as part of a belt or a strap.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

### Further Advantages

With reference to the foregoing embodiments, a skilled person will appreciate the cover designs of the present invention as alternatively described herein can help minimise the main issues in the past which made such passive diversion of digestive contents unattractive. Those main issues were:
i. Leak around the passive diversion tube-such leaks can be contained and/or absorbed.
ii. Expulsion of the tube by peristalsis of the proximal (upstream) bowel limb or accidental removal—the easy-to-use covers may assist in keeping a connecting device as described in place.
iii. Stasis/blockage of contents in the passive space—the flexible nature of the devices as described (and/or cap where present) can assist in removing blockages.

The described embodiments of the device are adapted to be inserted into a stoma(s). A device which is adapted to be inserted rather than one which is simply positioned over a surgically created orifice, like a stoma is advantageous.

The described device limits the likelihood of leaks as inflow and outflow of the digestive contents between the device and a patient occurs within the body of a patient (i.e. the legs 2106, 2108, 3001 are inserted into the stoma).

The device with leg(s) is adapted to be inserted into normal stomas which are generally everted (where the stoma is protruding outwards through the patient's skin to direct digestive contents outwards).

The device may also be particularly useful with stomas with problems such as when they are retracted or prolapsed.

Where a patient has a retracted stoma, where the stoma is below the skin surface, or lies flat against the skin surface (not everted), the legs of the device guide digestive contents away from the patient's skin to decrease the likelihood of leaks. Traditionally these stomas are problematic as they commonly lead to leaks and damage the surrounding skin.

The device may also be particularly beneficial for patients with a prolapsed stoma 6 where the intestinal tract lining telescopes out through the skin opening as illustrated in FIG. 19. A prolapsed stoma 6 may be reduced or reversed when the device is inserted through the stoma as the device exerts a constant low downward pressure onto the stoma. The device when inserted, prevents the stoma from being able to prolapse out again.

In some configurations, the cap or shell is coupled the plate structure 2900, 3300 or is adhered/pressed against a patient's body, so that the device is anchored to help keep the leg(s) inserted within the stoma. Preferably, the shell is rigid or semi-rigid.

The described devices may also be useful, as it acts like an adaptor to other components (e.g. a stoma bag) for imperfectly situated stomas, such as a stoma lying at a skin crease or in skin folds in the case of obesity, or other areas of scarring or deformity.

This described devices are particularly useful for patients with stomas which are prone to blocking, have a swollen bowel, or when the stoma aperture through the abdominal wall is too tight. In these cases, the device serves as an intestinal tract straightener or conduit for digestive contents to flow through the narrowing or site of obstruction.

This passive device is advantageous as it is a simple and effective device. The described device improves the convenience of inserting a device into a stoma. A simple to use device can increase compliance with therapy.

The driving force between the proximal and distal intestinal opening is provided by the flow pressure, and an external pump is generally unnecessary. A simple device as described, reduces the likelihood of mechanical failure.

The device also may be easily cleaned/sanitised in some configurations for possible reuse.

The described device comprises specific features which are adapted to make the device simple and easy to use, so that either a medical professional or a patient may be able to use the device.

An advantage of connecting device is that it is a partially external component, and is low-impact in nature. The connecting device is significantly easier to use and less burdensome than wearing a traditional stoma bag.

Patients may find the connecting device attractive as in the preferred configurations the device is portable, and may be self-operated and/or managed at home. This is beneficial as some hospital resources can be saved as the patient can manage their own device.

There is no need to worry about emptying a stoma bag. The connecting device may help enable patients' independence and improve their quality of life. There is no risk of stoma bag leaks, which sometimes occur when the bags become overfull.

To those skilled in the art to which the invention relates, many changes in construction and widely differing embodiments and applications of the invention will suggest themselves without departing from the scope of the invention.

1. A connecting device for connecting a surgically created first orifice with a surgically created second orifice and transferring contents from the surgically created first orifice to the surgically created second orifice, the device comprising:
a first elongate tubular member configured to, in use, be inserted into the surgically created first orifice,
a second elongate tubular member configured to, in use, be inserted into the surgically created second orifice,
an intermediate hollow body member having a body inlet and a body outlet, and
wherein the first elongate tubular member is in fluid communication with and extends away from the body inlet, the first elongate tubular member having an inlet located at a distal end away from the intermediate hollow body member, and
wherein the second elongate tubular member is in fluid communication with and extends away from the body outlet, the second elongate tubular member having an outlet located at a distal end away from the intermediate hollow body member, and
wherein the intermediate hollow body member extends between the first and second elongate tubular members, and
wherein the intermediate hollow body member includes a body opening, and the connecting device includes a cap for closing said body opening to complete a sealed pathway between the first elongate tubular member and the second elongate tubular member for passive transfer of contents from the first orifice to the second orifice, and
wherein said device further includes a shell configured to, in use, encompass and define a volume of space surrounding said surgically created first and second orifices, and wherein said volume of space is isolated from said pathway between the first elongate tubular member and the second elongate tubular member, so that the contents being transferred from the surgically created first orifice to the surgically created second orifice do not leak into the surrounding volume of space.

2. A connecting device as claimed in claim 1, wherein the shell is rigid or semi-rigid and adapted to exert a downward pressure to keep the first and second elongate tubular members within the surgically created first and second orifices when the device is attached to a patient.

3. A connecting device as claimed in claim 1, wherein said cap comprises a pathway seal on an inner surface of the cap adapted to seal the body opening.

4. A connecting device as claimed in claim 1, wherein said cap is separate and removable and when removed allows access to an internal surface of the intermediate hollow body member.

5. A connecting device as claimed in claim 1, wherein said cap and said shell are integrally formed.

6. A connecting device as claimed in claim 1, wherein the intermediate hollow body member and the first and second elongate tubular members are integrally formed.

7. A connecting device as claimed in claim 1, wherein the first and second elongate tubular members are separate and removable from the intermediate hollow body member.

8. A connecting device as claimed in claim 1, wherein the first and second elongate tubular members are flexible and resilient.

9. A connecting device as claimed in claim 1, wherein said device further comprises a plate structure for removable attachment to skin of a patient and around the surgically created first and second orifices, the plate structure comprising:
a first side and a second side that are opposite to one another, and the first side of the plate structure is configured to be attached to a skin of a patient using an adhesive; and
the second side of the plate structure is configured to be coupled to the shell.

10. A connecting device as claimed in claim 9, wherein the coupling of the shell to the plate structure is located around the perimeter of the shell.

11. A connecting device as claimed in claim 10, wherein the coupling of the shell to the plate structure comprises complementary features in the form of a groove and a complementary protrusion.

12. A connecting device as claimed in claim 9, wherein the coupling of the shell to the plate structure forms a watertight coupling.

13. A connecting device as claimed in claim 1, wherein the shell comprises an aperture to allow draining of digestive contents.

14. A connecting device as claimed in claim 7, wherein the shell further comprises a flat rim extending from a perimeter of the shell, the flat rim having an underside configured to sit against a patient's skin, and
wherein the underside of the flat rim comprises an adhesive to adhere the shell to the patient's skin.

15. A connecting device as claimed in claim 1, wherein silicone, rubber, latex or plastic form one or more of the:
a) First elongate tubular member,
b) second elongate tubular member,
c) intermediate hollow body member,
d) longitudinal guide member.

16. A method of connecting a surgically created first orifice with a surgically created second orifice, the method comprising:
providing a connecting device as claimed in claim 1,
inserting the inlet of the first elongate tubular member into the surgically created first orifice and the outlet of the second elongate tubular member into the surgically created second orifice so that the surgically created first and second orifices are connected by the device for passive transfer of contents from the surgically created first orifice to the surgically created second orifice.

17. A method of connecting a surgically created first orifice with a surgically created second orifice as claimed in claim 1, wherein the method further comprises a step of positioning and adhering a first side of a membrane of the plate structure to the skin of a patient.

18. A method of connecting a surgically created first orifice with a surgically created second orifice, wherein the method includes the steps of:
positioning a shell as claimed in claim 1 over the surgically created first and second orifices to substantially encompass and define a volume of space surrounding said surgically created first and second orifices, and
coupling the shell to the plate structure by coupling the plate coupling member to the shell coupling member.

19. A method of connecting a surgically created first orifice with a surgically created second orifice as claimed in claim 1, wherein the method further includes the step of positioning the cap over the body opening to close the pathway between the first elongate tubular member and the second elongate tubular member.
